(19) United States
(12) Patent Application Publication
(10) Pub. No.: US 2017/0333494 A1
(43) Pub. Date: Nov. 23, 2017

(54) PROBIOTIC THERAPEUTIC APPLICATIONS

(71) Applicants: GlaxoSmithKline Intellectual Property Development Limited, Brentford, Middlesex (GB); National Institutes of Health, Bethesda, MD (US)

(72) Inventors: David A. WILFRET, Research Triangle Park, NC (US); Jesse Daniel KEICHER, Research Triangle Park, NC (US); Helene Fischer ROSENBERG, Bethesda, MD (US)

(21) Appl. No.: 15/525,329

(22) PCT Filed: Nov. 9, 2015
(86) PCT No.: PCT/US15/59662

§ 371 Date: May 9, 2017

Related U.S. Application Data
(60) Provisional application No. 62/077,534, filed on Nov. 10, 2014.
(60) Provisional application No. 62/247,333, filed on Oct. 28, 2015.

(51) Int. Cl. A61K 35/747; A61M 15/00; A61K 9/00; A61K 9/16

(52) U.S. Cl. CPC A61K 35/747; A61K 9/1623; A61M 15/00; A61K 9/0043; A61K 9/1617; A61K 9/0075; A61M 2202/064

(57) ABSTRACT

Aspects of the present invention are related to the use of Lactobacillus species in a composition for respiratory administration to prevent the pathogenic inflammatory sequelae of respiratory virus infections.

### STATEMENT AS TO RIGHTS TO INVENTIONS MADE UNDER FEDERALLY SPONSORED RESEARCH AND DEVELOPMENT

This invention was created in the performance of a Cooperative Research and Development Agreement with the National Institutes of Health, an Agency of the Department of Health and Human Services. The Government of the United States has certain rights in this invention.

### TECHNOLOGY FIELD

Aspects of the present invention relate to novel therapeutic compositions for the administration of one or more strains of probiotic bacteria to a subject to treat, ameliorate, or lessen the severity thereof, and/or to prevent infectious disease, and in particular, for the treatment and/or prevention of respiratory infections.

### BACKGROUND

Probiotic bacteria are defined as live microorganisms which, when administered in adequate amounts beneficially affect the host. Lactobacilli and Bifidobacteria are the most frequently used bacteria in probiotic products. These bacteria are generally regarded as safe, as are probiotics based on these organisms.

Oral intake of different probiotic bacteria has been shown to have clinical benefits in various physiologic or pathologic situations. The most clear cut effects have been shown in diarrhea caused by antibiotic therapy or rotavirus infection. There are also studies showing positive clinical effects in inflammatory bowel disease, atopic dermatitis and hypercholesterolemia after oral intake of probiotic bacteria. The mechanisms by which probiotic bacteria contribute to these clinical improvements are presently not well understood.

Acute respiratory infections affecting the upper or lower respiratory tract are among the most common health problems among children and the elderly, although the incidence is high in all age groups. These respiratory infections cause a multitude of health care visits and hospitalizations every year as well as non-attendance at day care centers, schools, and jobs. In some instances, respiratory infections may result in premature death. However, the majority of respiratory tract infections are mild, self-limiting viral upper respiratory infections, also known as the “common cold,” most caused by strains of Rhinovirus.

Uncomplicated respiratory infections are widely misdiagnosed and often treated by antibiotics. This contributes to the overuse of antibiotics and simply adds to the development of multi-drug resistant bacteria as antibiotics do not provide efficacy for viral infections. Very few effective medications have been developed against viral infections.

Two marketed antiviral drugs are effective against influenza viruses but are hampered by limited versatility. Efficacy requires strict compliance to administration of the drug within 24 hour of infection. Beyond influenza, very few options exist for the prevention or the mitigation or relief of the symptoms caused by other common respiratory viruses. The development of a simple, safe and proven effective means to effect respiratory tract infections and/or the clinical sequelae including the inflammatory pathology of respiratory tract infections remains a major unmet medical need.

### SUMMARY

An embodiment of the present invention related to pharmaceutical composition comprising composite particles comprising Lactobacillus and an excipient.

An additional embodiment of the present invention relate to an inhaler comprising a pharmaceutical composition comprising dry powder composite particles comprising of Lactobacillus and an excipient, wherein the composite particles have a mass median aerodynamic diameter (MMAD) ranging from about 20 μm to about 30 μm.

An additional embodiment relates to an intranasal dry power delivery device comprising a pharmaceutical composition comprising of dry powder composite particles comprising Lactobacillus and an excipient, wherein the composite particles have a mass median aerodynamic diameter (MMAD) ranging from about 20 μm to about 30 μm.

A further embodiment relates to a method of preventing or treating a viral infection in a subject comprising administering to the subject a composition comprising one or more species of Lactobacillus bacteria.

A further embodiment relates to a method of preventing or treating a viral infection in a subject comprising administering to the subject a composition comprising a single species of Lactobacillus bacteria.

Another embodiment relates to a method of preventing or treating a viral infection in a subject comprising administering to the subject a composition comprising of the species of Lactobacillus plantarum bacteria or a strain thereof.

Another embodiment relates to a method preventing or treating a viral infection in a subject comprising administering to the subject a composition comprising a single strain of Lactobacillus plantarum selected from the group consisting of ATCC 10241, ATCC 14431, ATCC 39268, ATCC 21028, ATCC 55324, ATCC 39542, ATCC 14917, ATCC 700211, ATCC BAA-793, ATCC 4008, ATCC 8014, ATCC 10012, ATCC 49445, ATCC 53187, ATCC 700210, ATCC BAA-171, DSMZ 10492, DSMZ 1055, DSMZ 12028, DSMZ 24624, DSMZ 2648, DSMZ 6872 and DSMZ 16365.

A further embodiment relates to a method preventing or treating a viral infection in a subject comprising administering to the subject a composition comprising a single strain of plant derived Lactobacillus plantarum selected from the group consisting of ATCC 10241, ATCC 14431, ATCC 55324, ATCC 39542, ATCC 14917, ATCC 700211, ATCC 53187, ATCC BAA-171, DSMZ 10492, DSMZ 24624, DSMZ 2648 and DSMZ 16365.

Another embodiment relates to a method of preventing or treating the symptoms due to a viral infection in a subject comprising administering to the subject a composition comprising of one or more species of Lactobacillus bacteria.

Another embodiment relates to a method of preventing or treating the symptoms due to a viral infection in a subject comprising administering to the subject a composition comprising a single species of Lactobacillus bacteria.

Another embodiment relates to a method preventing or treating the of symptoms due to a viral infection in a subject comprising administering to the subject a composition comprising a single species of Lactobacillus bacteria wherein the species is Lactobacillus plantarum bacteria or a strain thereof.

Another embodiment relates to a method preventing or treating the of symptoms due to a viral infection in a subject comprising administering to the subject a composition comprising a single strain of Lactobacillus plantarum selected from the group consisting of ATCC 10241, ATCC 14431, ATCC 39268, ATCC 21028, ATCC 55324, ATCC 39542, ATCC 14917, ATCC 700211, ATCC BAA-793, ATCC 4008, ATCC 8014, ATCC 10012, ATCC 49445, ATCC 53187, ATCC 700210, ATCC BAA-171, DSMZ 10492, DSMZ 1055, DSMZ 12028, DSMZ 24624, DSMZ 2648, DSMZ 6872 and DSMZ 16365.

Another embodiment relates to a method preventing or treating the of symptoms due to a viral infection in a subject comprising administering to the subject a composition comprising a single strain of plant derived Lactobacillus plantarum selected from the group consisting of ATCC 10241, ATCC 14431, ATCC 55324, ATCC 39542, ATCC 14917, ATCC 700211, ATCC 53187, ATCC BAA-171, DSMZ 10492, DSMZ 24624, DSMZ 2648 and DSMZ 16365.

Another embodiment relates to a method of treating a viral infection in a subject comprising administering to the subject a composition comprising one or more strains of Lactobacillus bacteria to suppress virus-induced inflammation.

Another embodiment relates to a method of treating a viral infection in a subject comprising administering to the subject a composition comprising one or more strains of Lactobacillus bacteria to suppress virus-induced cytokine induction.

Another embodiment relates to a method of preventing or treating a secondary respiratory bacterial infection following an initial respiratory viral infection in a subject comprising administering to the subject a composition comprising one or more species of Lactobacillus bacteria.

Another embodiment relates to a method of preventing or treating a secondary respiratory bacterial infection following an initial respiratory virus infection in a subject comprising administering to the subject a composition of one species of Lactobacillus bacteria consisting of Lactobacillus plantarum.

An addition aspects of the present invention relates to a pharmaceutical composition comprising: from about 40 to about 60% Lactobacillus bacteria of the composition; from about 40 to about 60% w/w trehalose; wherein said Lactobacillus bacteria is heat inactivated; and wherein said Lactobacillus bacteria is whole cell.

Another aspect of the present invention relates to a method of treating at least one symptom of a cold or flu comprising administering to the subject a composition comprising one or more species of Lactobacillus bacteria.

### BRIEF DESCRIPTION OF THE DRAWINGS

FIG. 1 illustrates the impact of L. plantarum (abbreviated throughout as “LP” or “Lp” or “Lac”) on survival of BALB/c mice in response to an otherwise lethal pneumonia virus of mice (PVM) infection. Here, a single intranasal inoculum of 50 μL at 2×10¹⁰ cells/mL of inactivated L. plantarum (Lp-F3) administered one day prior to virus challenge results in 100% survival (**p<0.01 log rank).

FIG. 2 illustrates that L. plantarum administered after PVM challenge also results in survival of BALB/c mice in response to an otherwise lethal PVM infection. Here, a single intranasal inoculum of 50 μL of 2×10⁹ cells/mL L. plantarum, heat-inactivated as described in Gabryszewski et al., 2011 [J. Immunol. 186: 1151-1161] (Lp-F0) administered to BALB/c mice on day +1 or on days +1 and +2 after PVM challenge also results in 100% survival (***p<0.001 log rank).

FIG. 3 illustrates the biochemical inflammatory responses of BALB/c mice that were inoculated intranasally with 50 μL of 2×10⁹ cells/mL heat-inactivated L. plantarum (Lp-F0) as described in Gabryszewski et al., 2011 [J. Immunol. 186: 1151-1161] on day +1 or on days +1 and +2 after PVM challenge (as in FIG. 2). Proinflammatory cytokines CCL2, CXCL10, and IL6 were detected by ELISA in lung homogenates at day +5. As shown, expression of proinflammatory cytokines are suppressed in mice with L. plantarum when administered as a single inoculum on day +1 (only) or once each on days +1 and +2 after PVM challenge compared to mice inoculated with diluent (PBS) alone (**p<0.01, Mann-Whitney U-test).

FIG. 4 illustrates virus recovery from lung tissue of BALB/c mice that were inoculated intranasally with 50 μL of 2×10⁹ cells/mL heat-inactivated L. plantarum (Lp-F0) on day +1 or on days +1 and +2 after PVM challenge (as in FIG. 2). Virus recovery (determined by qRT-PCR; Percopo et al., 2014b) at day +5 is reduced ˜5-15 times, respectively compared to control mice that were inoculated with diluent only (*p<0.05, **p<0.01, Mann-Whitney U-test).

FIG. 5A illustrates lung tissue from BALB/c mice that were inoculated intranasally with diluent only on days +1 and +2 after PVM challenge (as in FIG. 2) and includes prominent alveolitis and congestion, indicating initial onset of edema. FIG. 5B illustrates lung tissue from BALB/c mice that were inoculated intranasally with 50 μL of 2×10⁹ cells/mL heat-inactivated L. plantarum, Lp-F0 on days 1 and 2 after PVM challenge as in FIG. 2 that exhibit substantially less inflammation.

FIG. 6 illustrates differential survival of BALB/c mice in response to priming with 50 μL of 2×10¹⁰ cells/mL live L. plantarum (Lp-F00) or PBS on days −14 and −7 followed by challenge with PVM 21 days later (on day +14) [figure redrawn from Garcia-Crespo et al., 2013, Antiviral Res. 97: 270-279]. Only mice that are primed with L. plantarum on days −14 and −7 survived PVM challenge, (**p<0.01 log rank).

FIG. 7 illustrates that profound suppression of virus-induced proinflammatory cytokines CCL2, CXCL10, and IL-6 is observed in response to priming with 50 μL of 2×10¹⁰ cells/mL live L. plantarum (Lp-F00) on days −14 and −7 and is associated with survival as shown in FIG. 6, (**p<0.01, Mann-Whitney U-test).

FIG. 8 illustrates the observation that BALB/c mice primed only once (on day −7 or day −14 alone) with 50 μL of 2×10¹⁰ cells/mL live L. plantarum (Lp-F00) do not survive in response to a subsequent PVM challenge on day +14 [figure redrawn from Garcia-Crespo et al., 2013, Antiviral Res. 97: 270-279], (**p<0.01 log rank).

FIG. 9 illustrates that suppression of proinflammatory cytokines is observed only in response to priming with 50 μL of 2×10¹⁰ cells/mL live L. plantarum (Lp-F00) on both days −14 and −7, the same priming regimen that is associated with full survival in response to PVM challenge. Conversely, the mice which were primed with L. plantarum only once (either on day −14 or on day −7 alone) did not survive PVM challenge (FIG. 7) nor did this L. plantarum priming regimen result in suppression of virus-induced proinflammatory cytokines CCL2, CXCL10, and IL-6 (**p<0.01, Mann-Whitney U-test).

FIG. 10 illustrates the observation that heat-inactivated L. plantarum (Lp-F4) used to prime the respiratory mucosa via the standard protocol (50 μL, 2×10¹⁰ cells/mL at days −14 and −7) also protects against the lethal sequelae of Influenza A/HK/68 (H3N2) challenge, (**p<0.01 log-rank).

FIG. 11 illustrates that a single inoculum of heat-inactivated L. plantarum (Lp-F4) 50 μL, 2×10¹⁰ cells/mL elicits full protection against the lethal sequelae of PVM in BALB/c mice through 7 days; protection is lost as early as 10 days in response to this single L. plantarum inoculum (**p<0.01 log-rank).

FIG. 12 illustrates that two inoculations (here, as indicated on days −7 and 0) of heat-inactivated L. plantarum 50 μL at 2×10¹⁰ cells/mL formulated either in PBS buffer (Lp-F3) or in PBS buffer containing 10% trehalose (Lp-F4), result in a dramatic increase in duration of protection over that observed in response to priming with one inoculum alone. Here, we observe full protection against the lethal sequelae of PVM is sustained through 42 days (longest duration tested) after the second L. plantarum inoculation (**p<0.01 log-rank).

FIG. 13 illustrates the importance of the interval between successive L. plantarum inoculations. Heat-inactivated L. plantarum (Lp-F4) inoculated in a volume of 50 μL, at 2×10¹⁰ cells/mL was administered on two consecutive days (days −1 and 0); protection against the lethal sequelae of PVM infection was sustained up to 10 days followed by a precipitous drop by day 21 (*p<0.05 log-rank). With doses remaining constant per inoculation, protection provided in response to two inoculations on two consecutive days is only slightly longer than that observed in response to a single inoculation (see FIG. 11). Despite receiving two inoculations of L. plantarum, in this case, on two consecutive days (days −1 and 0), mice did not achieve the extended duration of protection that was observed when the two inoculations were administered one week apart. (see FIG. 12).

FIG. 14 illustrates that the full protection from a lethal virus challenge can be sustained in BALB/c mice for at least 7 months (longest duration tested) by the administration of L. plantarum via once or twice monthly intranasal inoculations. In this experiment, mice initially received a loading protocol of heat-inactivated L. plantarum, Lp-F3 (50 μL at 1.3×10⁹ cells/mL) or PBS consisting of two doses, once on day −7 and once on day 0, followed by repeat once monthly inoculations (with Lp-F3 or PBS) thereafter for 6 months. One month (28 days) after the last L. plantarum inoculation, these mice were challenged with a fully lethal dose of PVM. Only the mice that received L. plantarum were protected (100% survival). Furthermore, mice received a loading protocol once on day −7 and day 0, followed by repeat twice monthly inoculations for 6 months achieved the same 100% survival against a subsequent fully lethal PVM challenge (**p<0.01 log-rank). These data demonstrate that the protective effect elicited by L. plantarum is not lost via tachyphylactic-type mechanisms and can be sustained with repeat dosings.

FIG. 15 illustrates that heat-inactivated L. plantarum (Lp-F2) induced protection of BALB/c mice against lethal PVM challenge is dose dependent. Here, it was found that 50 mL of 2×10⁹ cells/mL (total dose equal to 1×10⁸ cells/mouse) was the minimum efficacious dose required to achieve 100% survival against a fully lethal PVM challenge in BALB/c mice.

FIG. 16 illustrates the efficacy of L. plantarum in a strict upper respiratory tract non-lethal infection model. As depicted, BALB/c mice were inoculated via strict intranasal protocol (2.5 microliter per each nare) with inactivated L. plantarum, Lp-F3 (2.5 microliter/nare at 1×10̂¹¹ cells/mL) followed by the strict intranasal challenge with H3N2 influenza virus. Here, mice received either two inoculations of L. plantarum one inoculation a week for two weeks or four inoculations of L. plantarum (Lp-F3), one inoculation a week for four weeks, prior to challenge with Influenza A/HK/68 (H3N2). Only the mice receiving L. plantarum were protected against H3N2 influenza-induced weight loss.

FIG. 17 illustrates the relative responses elicited in an in vitro signaling assay by L. plantarum Lp-F1 and Lp-F2 (final concentration 1×10⁸ cells/mL) by HEK-293 cells expressing specific pattern recognition receptors (PRRs) in vitro. L. plantarum specifically activated toll like receptor 2 (TLR2) and nucleotide binding oligomerization domain-containing protein 2 (NOD2) signaling by as much as 20-fold and 6-fold, respectively, over diluent control in stably transfected HEK293 cells in vitro, as shown (*p<0.05, **p<0.01 log-rank).

FIG. 18 illustrates L. plantarum Lp-F1 and Lp-F2 (final concentration of 1×10⁸ cells/mL) does not activate C-type lectin (CLR), dectin 1a or dectin 1b pattern recognition receptors (PRRs) in vitro.

FIG. 19 illustrates L. plantarum Lp-F1 and Lp-F2 (final concentration of 1×10⁸ cells/mL) induced both NF-κB and IRF pathways in the THP human monocyte cell line at 8 to 12-fold over baseline in vitro.

FIG. 20 illustrates that gene-deleted pattern recognition receptor, toll-like receptor 2 (TLR2^{-/-} mice) mice and gene-deleted nucleotide-binding oligomerization domain-containing protein 2 (NOD2^{-/-} mice) mice respond as do to their wild type (C57BL/6) counterparts to priming with L. plantarum (Lp-F0, 50 μL of 2×10¹⁰ cfu/mL) and are protected against subsequent challenge with PVM (***p<0.001; *p<0.05, log-rank).

FIG. 21 illustrates that TLR2^{-/-} mice respond to L. plantarum (Lp-F0) priming (see FIG. 20) with reduced virus recovery from lung tissue (*p<0.05, Mann-Whitney U-test).

FIG. 22 illustrates TLR2^{-/-} mice respond to L. plantarum (Lp-F0) priming (see FIG. 20) with a prominent suppression of cytokines CCL2, CXCL10, and IL-6 (**p<0.01, Mann-Whitney U-test).

FIG. 23 illustrates that TLR2^{-/-} and NOD2^{-/-} mice respond as do their wild type (C57BL/6) counterparts and remain responsive to L. plantarum (Lp-F0) administered to the respiratory mucosa after PVM challenge and are protected against the lethal sequelae of PVM challenge (*p<0.05, log-rank; **p<0.01).

FIG. 24 illustrates NOD2^{-/-} mice inoculated with L. plantarum (Lp-F0) on days +1 and +2 after PVM challenge demonstrate reduced virus recovery from lung tissue.

FIG. 25 illustrates NOD2^{-/-} mice inoculated with L. plantarum (Lp-F0) on days +1 and +2 after PVM challenge demonstrate prominent suppression of cytokines CCL2, CXCL10, and IL-6 (*p<0.05, **p<0.01, Mann-Whitney U-test).

FIG. 26 illustrates that IFNαβR^{-/-} mice respond as do their wild type (C57BL/6) counterparts and remain responsive to L. plantarum (Lp-F0) administered to the respiratory mucosa after PVM challenge and are protected against the lethal sequelae of PVM challenge (*p<0.05, **p<0.01 log-rank).

FIG. 27 indicates the percent of whole cells remaining in L. plantarum preparations following the heat inactivation conditions as described in Gabryszewski et al. 2011 [J. Immunol. 186: 1151-1161] (Lp-F0) compared to the optimized inactivation conditions used to generate L. plantarum Lp-F3 and Lp-F4 (*p<0.05, log-rank).

FIG. 28 illustrates the finding that the cryoprotectant glycerol reduces the efficacy of L. plantarum induced protection against lethal PVM infection. BALB/c mice were inoculated on days −14 and −7 with L. plantarum (Lp-F0)) 50 μL, 2×10¹⁰ cells/mL formulated either with or without 20% glycerol followed by PVM on day +35. As shown, the addition of glycerol in the formulation reduces the efficacy of L. plantarum at an otherwise fully protective dose.

FIG. 29 illustrates the discovery that the cryoprotectant, consisting of 10% trehalose buffer solution does not have an apparent impact on the efficacy of L. plantarum-mediated protection. As shown, BALB/c mice were inoculated intranasally with various L. plantarum preparation either in PBS buffer (Lp-F3) or L. plantarum in PBS buffer with 10% trehalose (Lp-F4) 50 μL, 2×10¹⁰ cells/mL on days −14 and −7 followed by PVM on day +35. Protection was elicited equally well by both L. plantarum preparations (**p<0.01, log-rank).

FIG. 30 depicts the nature of trehalose as an effective cryopreservative. A 10% trehalose solution prevents cell lysis and cell aggregation/disaggregation, and thus, effectively maintains the physical morphology of the heat-inactivated bulk drug substance when frozen for purposes of storage and shipping.

FIG. 31 depicts the particle size distribution and SEM image of a representative example of L. plantarum in a 10% trehalose buffer solution (Lp-F4) as a spray dried drug product.

FIG. 32 depicts minimal disruption of the whole cell L. plantarum drug substance in the final spray dried drug product following the sequence of initial manufacturing, heat-inactivation, frozen shipping, thaw, and spray drying manufacturing. Minimal lysis (1.1%) was observed in this representative example of the final spray dried drug product made from Lp-F4.

FIG. 33 illustrates that, similar to the wild-type (BALB/c) counterparts, mice devoid of interleukin-10 (IL-10^{-/-} mice) remain responsive to L. plantarum, Lp-F0 (10⁹ cfu/mouse on days +1 and +2) administered to the respiratory mucosa after PVM challenge and are protected against the lethal sequelae of PVM challenge (***p<0.001, log-rank).

FIG. 34 illustrates that, similar to wild-type (BALB/c) mice, L. plantarum (10⁹ cfu/mouse on days 1 and 2 after virus challenge), administered to IL-10^{-/-} mice results in diminished virus recovery from lung tissue (***p<0.001, Mann-Whitney U-test).

FIG. 35 illustrates that, similar to the wild-type (BALB/c) counterparts, L. plantarum (10⁹ cfu/mouse on days 1 and 2 after virus challenge), administered to IL-10^{-/-} mice results prominent suppression of cytokines CCL2, CXCL10, and IL-6 (**p<0.01, Mann-Whitney U-test).

FIG. 36 illustrates that, similar to the wild-type (C57BL/6) mice, L. plantarum (10⁹ cfu/mouse on days 1 and 2 after PVM challenge) administered to mice devoid of interleukin-17A (IL-17A^{-/-} mice), are protected against the lethal sequelae of PVM challenge (**p<0.01, log-rank;***p<0.001, log-rank).

Table 1 illustrates the significant (0.05, except where noted*) differential gene expression (>1.5-fold) of virus-induced soluble proinflammatory mediators in response to priming with L. plantarum. BALB/c mice were inoculated intranasally with L. plantarum (LP-F00) or diluent control (pbs/bsa; PBS) on days −14 and −7, followed by inoculation with pneumonia virus of mice (PVM; 0.2 TCID₅₀ units/50 μL) or vehicle (VEH; pbs+0.1% bsa) control on day +14. Featured is the differential expression of 31 soluble proinflammatory mediators, a subset of the 839 differentially expressed transcripts detected by whole genome microarray from lung tissue evaluated on day +19, 5 days after inoculation of PVM. Among those most profoundly suppressed include IL-6, CCL2, CXCL10, CXCL2 and CXCL11, which undergo 105, 11, 14, 20 and 21-fold reduced expression, respectively.

### DETAILED DESCRIPTION

Acute respiratory infections affecting the upper or lower respiratory tract are among the most common health problems among children and the elderly, though the incidence is high in all age groups. These respiratory infections cause multitude of health care visits and treatment periods in hospitals every year as well as non-attendance in day care centers and jobs. In most drastic cases, the respiratory infections may cause premature death of the elderly. However, the majority of respiratory tract infections are mild, self-limiting viral upper respiratory infections, also known as the common cold. A majority of colds are caused by a viral strain of Rhinovirus however, respiratory syncytial virus (RSV), metapneumovirus, parainfluenza virus, adenovirus, and influenza contribute to the vast number of respiratory viral infections each year. In the present invention, otherwise un-manipulated, heat inactivated, whole cell Lactobacillus plantarum, when delivered directly to respiratory mucosa suppresses the proinflammatory pathology and negative sequelae associated with viral respiratory tract infections.

Presently, there are no effective vaccines or drugs available to treat the vast majority of viral respiratory infections. Although there are some effective medications and vaccinations have been successful in reducing the incidence of influenza infection, there are no effective vaccines or medications available against the majority of other common respiratory viruses with the exception of the mAb Synagis® (Palivizumab) which is used to prevent RSV. However, at this time, palivizumab use is limited to select high risk population including premature infants, children 24 months or less with bronchopulmonary dysplasia (BPD) and/or hemodynamically significant congenital heart disease (CHD). However, these are not the only children at risk for severe infection [Hall et al., 2009 N. Engl. J. Med. 360: 588-598]. Thus, more widely applicable effective agents for preventing or mitigating the inflammatory sequelae of severe respiratory infections represent an unmet medical need.

Intranasal administration of Lactobacillus species has been evaluated in mouse models of severe respiratory virus infection. Of these studies, Rosenberg and colleagues [Gabryszewski et al., 2011 J. Immunol. 186: 1151-1161] have demonstrated sustained protection against lethal respiratory virus infection, specifically, protection against the lethal sequelae of pneumonia virus of mice (PVM) virus infection lasting up to 6 months after Lactobacillus administration. Likewise, Rosenberg and colleagues [Gabryszewski et al., 2011 J. Immunol. 186: 1151-1161; Garcia-Crespo et al., 2013 Antiviral Res. 97: 270-279] have identified an association of profound cytokine suppression concurrent with survival.

Rosenberg and colleagues [Gabryszewski et al., 2011 J. Immunol. 186: 1151-1161; Garcia-Crespo et al., 2013 Antiviral Res. 97: 270-279; Percopo et al., 2014a J. Immunol. 192: 5265-5272] have shown that priming of the respiratory mucosa with live or heat-inactivated L. plantarum results in a reduction in airway pathology associated with survival in response to an otherwise lethal challenge with the respiratory virus pathogen, pneumonia virus of mice (PVM). PVM is a natural rodent pathogen that is in the same virus Family (Paramyxovirdae) and genus (Pneumovirus) as the human pediatric pathogen, respiratory syncytial virus (RSV), an important respiratory pathogen of infants and children for which there is currently no vaccine [Rudraraju et al., 2013 Viruses 5: 577-594]. However, unlike RSV, PVM undergoes robust replication in mouse lung tissue and replicates the pathophysiology of the more severe forms of human RSV disease in inbred strains of mice [Rosenberg & Domachowske, 2008 Immunol. Lett 118: 6-12; Dyer et al., 2012 Viruses. 4; Bem et al., 2011 3494-3510 Am J Physiol Lung Cell Mol Physiol. 301:L148-L156; Rosenberg & Domachowske, 2012 Curr Med Chem 19: 1424-1431]. In contrast, neither human respiratory syncytial virus (hRSV) or human rhinovirus (hRV) are capable of undergoing multiple replication cycles in rodent hosts and neither pathogen generates significant pathology or endpoints relevant to human disease. As such, PVM represents a more informative experimental model in which to evaluate responses to a targeted anti-inflammatory therapeutic agent in experiments carried out in inbred mice.

The inflammatory response to respiratory virus infection can be complex and refractory to standard therapy. Lactobacillus species L. plantarum or L. reuteri, when used to prime the respiratory tract, are highly effective at suppressing virus-induced inflammation and protecting against lethal disease. Rosenberg and colleagues [Gabryszewski et al., 2011 J. Immunol. 186: 1151-1161] outlined an experimental protocol for intranasal administration of live or heat-inactivated Lactobacillus species that results in the prevention of the lethal sequelae of respiratory viral infection. On days −14 and −7 (time-points prior to virus inoculation at day 0), 8 week old BALB/c mice were inoculated intranasally with either 10⁹ CFU or cells L. plantarum, 10⁹ CFU or cells L. reuteri, or phosphate buffered saline (PBS) with 1% bovine serum albumin (BSA), hereafter known as PBS/BSA, or vehicle control, each inoculum delivered in a 50 μL volume. On day 0, all mice were inoculated with an otherwise lethal dose of pneumonia virus of mice (PVM). BALB/c mice that were previously inoculated intranasally with live or heat-inactivated L. plantarum or live L. reuteri (hereafter known as “primed”) were completely (100%) protected from an otherwise lethal PVM infection. Rosenberg and colleagues also found that C57BL/6 mice could be protected against lethal PVM infection via priming with L. plantarum or L. reuteri using this protocol [Garcia Crespo et al., 2013 Antiviral Res. 97: 270-279; Percopo et al., 2014a J. Immunol. 192: 5265-5272].

The protection elicited via this protocol is in some instances, sustained. When 8 week old BALB/c mice were primed with live L. plantarum on day −14 and day −7 as described above and challenged with an otherwise fully lethal inoculum of PVM not at day 0, but at +91 days (3 months after initial priming), 60% of the mice survived [Gabryszewski et al., 2011 J. Immunol. 186: 1151-1161]. When the PVM challenge was delayed until +153 days, or 5 months after the initial L. plantarum priming, 40% of the mice survived a PVM infection that was fully lethal to the unprimed mice [Garcia-Crespo et al., 2013 Antiviral Res. 97: 270-279].

Protection against the lethal sequelae of PVM infection cannot be reproduced by oral intake of live L. plantarum. In an experimental trial, in which 8 week old BALB/c mice received 10⁹ cells L. plantarum/mL (approximately 5×10⁹ cells/day) in drinking water for 2 weeks prior to inoculation with PVM on day 0, and ongoing after PVM inoculation, no protection was observed, and all mice succumbed to lethal infection between days 7 to 11 after inoculation [Percopo et al., 2014a Methods Mol. Biol. 1178: 257-266)].

The degree of morbidity and mortality experienced in response to respiratory virus pathogens depends largely upon the extent to which inflammation is elicited by the pathogen in the specific host [reviewed in Mukherjee & Lukacs 2013 Curr Top Microbiol Immunol. 372: 139-154; Hallstrand et al., 2014 Clin Immunol. 151: 1-15]. Of interest, inflammation can persist even after virus replication has been brought under control with effective replication inhibitors, such as ribavirin [Bonville et al., 2003 J. Virol. 77: 1237-1244; Bonville et al., 2004 J. Virol. 78: 7984-7989]. Thus, the importance of inflammation to the outcome of respiratory virus infections has motivated an exploration of targeted anti-inflammatory therapies.

The differential expression of mRNA transcripts encoding proinflammatory mediators in lung tissue and differential expression of immunoreactive proinflammatory mediators in the airways in response to Lactobacillus priming has been explored in PVM-infected mice. Diminished expression (both mRNA and protein) of proinflammatory chemokines CCL2, CXCL10 and IL-6 is a prominent biomarker associated with survival in response to Lactobacillus-mediated priming [Percopo et al, 2015, ms in review]. Microscopic pathology of lung tissue from Lactobacillus-primed, PVM infected mice has been examined. Lung tissue from PVM-infected mice that were not primed with Lactobacillus exhibit a profound alveolitis, with widespread, diffuse granulocyte recruitment and early-onset edema. In contrast, the lung tissue of L. plantarum-primed, PVM-infected mice exhibited minimal inflammation peripherally, consistent with profound suppression proinflammatory cytokines and chemokines. Diminished recruitment of proinflammatory neutrophils was confirmed and evaluated quantitatively [Gabryszewski et al., 2011 J. Immunol. 186: 1151-1161].

Virus recoveries from lung tissue of L. plantarum, L. reuteri, and control-primed, PVM infected mice were determined by quantitative reverse-transcriptase polymerase chain reaction targeting the PVM small hydrophobic (SH) gene (qRT-PCR; Percopo et al., 2014b). While some differences in virus titer were detected, they were not profound, and at peak virus titer (day 5 after PVM inoculation), no significant differences were detected when comparing control-primed mice (which do not survive virus infection) to those primed with L. reuteri (which do survive virus infection; Gabryszewski et al., 2011 J. Immunol. 186: 1151-1161). In an effort to explore this issue further, the PVM inoculum administered to control-primed mice was reduced so that virus titer recovered from control-primed mice at peak (day 5) would be indistinguishable from those recovered from L. plantarum-primed mice. In this experimental design, the peak virus titers recovered at day 5 were statistically equivalent to one another, yet the L. plantarum-primed mice survived, and the control-primed mice all succumbed to the lethal PVM infection [Gabryszewski et al., 2011 J. Immunol. 186: 1151-1161]. Thus, it is clear that virus recovery alone cannot predict the outcome of an ongoing lethal infection. [Gabryszewski et al., 2011 J. Immunol. 186: 1151-1161].

Rosenberg and colleagues [Garcia-Crespo et al., 2013 Antiviral Res. 97: 270-279] performed a series of studies designed to examine the direct responses of the airways and lung tissue to Lactobacillus-priming prior to virus challenge. Among their findings were elevated levels of proinflammatory mediators CXCL1, CCL3, IL-17A, and TNF-α which were detected in lung tissue within 24 h after the first intranasal inoculation with live L. reuteri. These responses were transient and cytokine levels returned to baseline levels within several days. In response to a second L. reuteri inoculation one week later, cytokine production was more robust and sustained. In addition to the aforementioned mediators, significantly elevated levels of CCL2 and CXCL10 were detected 24 h after the second L. reuteri inoculation. Despite the induction of proinflammatory mediators, no immunoreactive antiviral cytokines, IFN-α or IFN-β were detected in lung tissue in response to priming with L. reuteri at any time points. Similarly, Lactobacillus-priming elicited only minimal production of IFNγ and there was no increase in the anti-inflammatory cytokine IL-10.

In an effort to explore the role of individual Lactobacillus components that might be eliciting protective responses, Rosenberg and colleagues [Garcia-Crespo et al., 2013 Antiviral Res. 97: 270-279] primed mice with L. reuteri genomic DNA (gDNA) in amounts 100-1000 fold greater than what would be inoculated in conjunction with live or inactivated bacteria. Priming with L. reuteri gDNA had only minimal and transient impact on virus recovery. Priming with L. reuteri gDNA had no impact on induction of the proinflammatory mediator, CCL3, and no impact on survival in response to PVM infection.

Similarly, mice were primed on days −14 and −7 with gram-positive peptidoglycan (PGN; 100 μg/mouse/inoculation, roughly equivalent to a PGN inoculum from 10⁹ bacteria.) This resulted in delayed mortality (median survival, t½=9.0 vs. 10.5 days, but it did not confer sustained survival such as that observed in response to priming with live L. reuteri. No significant protection against lethal PVM challenge was observed in response to priming with 10 or 50 μg PGN/mouse/inoculation.

Rosenberg and colleagues [Gabryszewski et al., 2011 J. Immunol. 186: 1151-1161] also examined Lactobacillus priming, survival, virus recovery and cytokine suppression in mice devoid of the “universal” TLR adapter, MyD88 (MyD88^{-/-}, mice on the C57BL/6 background). Although patterns of virus recovery and cytokine suppression (CCL2, CXCL10, CXCL1) differed significantly from those observed in C57BL/6 wild-type counterparts, Lactobacillus priming on days −14 and −7 followed by virus inoculation on day 0 resulted in protection against lethal PVM infection.

Several published studies have addressed the impact of oral administration of Lactobacillus species as potential, if only marginally effective, prevention against respiratory virus infection. The Cochrane Collaboration [Hao et al., 2011 Lett. Appl. Microbiol. 50: 597-602] which reviewed clinical trials of oral intake of various probiotics including, but not limited to Lactobacillus plantarum, led to the conclusion that probiotics were safe and adverse effects were minor. Furthermore, the compiled results suggested that probiotic therapy may provide benefit over placebo in terms of the episode rate of acute upper respiratory tract infections and likewise in terms of reducing the extent of antibiotics used for this diagnosis, although the results did not show any benefit in terms of duration of episodes of acute upper respiratory tract infection. However, overall, these outcomes are relatively minor and have only a minor impact on health and well-being compared the results that we have obtained via priming the respiratory mucosa directly.

Unlike what has been reported to take place in the gastrointestinal tract, intranasal administration of Lactobacillus does result in the colonization in respiratory tract [Garcia-Crespo et al., 2013 Antiviral Res. 97: 270-279]. Following intranasal inoculation, live colony forming units (cfu) of L. reuteri were detected in lung tissue homogenates, but they were cleared within 24 hrs of administration, with no evidence of bacterial replication in situ. Genomic DNA from L. reuteri could be detected for up to 48 h by qPCR after bacterial inoculation, however no L. reuteri genomic DNA was detectable after this time point. Similarly L. reuteri peptidoglycan was detected in lung tissue by silkworm-larvae melanocyte assay for 24 hrs only after the first of two inoculations.

It is clear from the results of these studies and the studies disclosed herein that the interactions of Lactobacillus with the local immune environment of the gastrointestinal mucosa are functionally distinct from what we observe in the respiratory tract. Among the most prominent differences, Lactobacillus-mediated protection from inflammatory sequelae has been attributed in large part to the actions of the anti-inflammatory cytokine, IL-10 [reviewed in Claes, et. al., 2011. Mol. Nutr. Food Res. 55: 1441-1453]. For example, Macho Fernandez and colleagues [2001 Gut 60: 1050-1059] showed that peptidoglycan derived from L. salivarius strain Ls33 served to protect mice from the inflammatory sequelae of chemical colitis via mechanisms that correlated with local production of IL-10. Similarly, Chen and colleagues [2005 Pediatr. Res. 58, 1185-119] found that inoculation of young mice with L. acidophilus stimulated IL-10 expression in conjunction with protection against colitis induced by the bacterial pathogen, Citrobacter rodentium. In a recent study by Bosch and colleges [2012 Lett. Appl. Microbiol. 54: 240-246], L. plantarum strains derived from the human gastrointestinal tract of potential probiotic interest were evaluated for, among other traits, their ability to induce production of IL-10. In contrast, as demonstrated in the examples presented herein, protection mediated by L. plantarum at the respiratory tract does not require IL-10, nor do we observe any difference in expression of biomarker cytokines when comparing the responses of IL-10−/− mice to their wildtype (BALB/c) counterparts.

Furthermore, as previously described [Garcia-Crespo, et. al., 2013. Antiviral Res. 97: 270-279; Percopo, et. al., 2014. J. Immunol. 192: 5265-5272], protection, or heterologous immunity elicited by L. plantarum at the respiratory tract may take place via mechanisms that are distinct from those observed in response to other bacterial species or in response to their isolated components. For example both Wiley and colleagues [2009 PLoS One 4(9):e7142] and Richert and colleagues [2012 Vaccine 30:3653-3665] reported that heterologous immunity against respiratory virus infections (including PVM) elicited by nanoparticles derived from the thermophilic bacteria M. jannaschii was directly dependent on accelerated local immunity directly dependent on the presence of B cells in bronchus associated lymphoid tissue (BALT); in contrast, it has been shown that heterologous immunity elicited by L. plantarum was fully functional in two independent strains of B cell deficient mice [Percopo, et. al., 2014. J. Immunol. 192: 5265-5272]. Likewise, as noted earlier, Schnoeller and colleagues [2014 Am. J. Respir. Crit. Care Med. 189: 194-202], recently reported that an attenuated preparation of Bordetella pertussis protected mice against clinical symptoms attributed to subsequent infection with RSV, a virus pathogen that is closely-related to PVM, via a mechanism dependent on production and activity of the proinflammatory cytokine, interleukin-17A. Interestingly, while L. plantarum inoculation alone results in production of IL-17A [Garcia-Crespo, et. al., 2013. Antiviral Res. 97: 270-279], in the examples presented herein, full protection against PVM mediated by L. plantarum administration in IL-17A gene-deleted mice is demonstrated.

In the examples presented herein, heterologous immunity elicited by L. plantarum in mice devoid of pattern recognition receptors, TLR2 and NOD2 is explored. While L. plantarum clearly interacts with these pattern recognition receptors (PRRs) and signals via TLR2 and NOD2 alone in in-vitro assays, it was found that mice with these individual gene deletions were fully protected in both priming and post-virus challenge protocols. The survival responses using priming protocols in TLR2−/− mice may have been anticipated to some extent given the aforementioned findings in MyD88−/− mice [Gabryszewski, et al., 2011. J. Immunol. 186:1151-1161]; however the survival responses and the concomitant suppression of cytokines in these mice was found to be analogous to their wild type (C57BL/6) counterparts. There may be cross-talk between TLR2 and NOD2 pathways [Wu, et al., 2015. Mol. Immunol. 64: 235-243; Zeuthen, et al., 2008. Immunology 124: 489-502; Borm, et al., 2008. Genes Immun. 9: 274-278; Netea, et al., 2005 J. Immunol. 174: 6518-6523; Pavot, et al., 2014. J. Immunol. 193: 5781-5785; Watanabe, et al., 2006. Immunity 25: 473-485].

There are to date only a few published studies that have examined the impact of Lactobacillus administered as an agent to prime the respiratory mucosa directly. Of these studies, ours is the only administration strategy that clearly results in a robust and sustained degree of protection against lethal respiratory virus infection (ie., significant survival at 3-5 months after priming), and likewise, the only study in which suppression of specific inflammatory mediators have been identified as biomarkers associated with Lactobacillus-mediated protection.

In addition to the aforementioned studies published by Rosenberg and colleagues in a recent publication, Park and colleagues [2013 PLoS One. 9: e75368] found that BALB/c mice subjected to intranasal inoculation at three time points—four days prior, one day prior and simultaneously with an otherwise lethal challenge with influenza A/PR8 (10⁸ cells L. plantarum DK119 per inoculation/mouse) were protected from severe weight loss and lethal sequelae characteristic of this infection. The authors did not explore any other intervals between priming and virus challenge, they did not evaluate any possibility of sustained protection nor did they examine the efficacy of inactivated L. plantarum. The authors did examine proinflammatory cytokines in the airways, but not via inoculation strategies that permit an evaluation of the relationship between Lactobacillus-mediated cytokine suppression and survival.

In an earlier study, Youn and colleagues [2012 Antiviral Res. 93: 138-143] examined the protective effects of both live and 3% formalin-inactivated Lactobacillus strains, including L. rhamnossus, L. brevis, and L. plantarum, against a lethal inoculum of Influenza A/NWS/33 (H1N1) also in BALB/c mice. Mice were inoculated once per day for 3 weeks (21 inoculations, each with 10⁸ cells) prior to virus challenge on day 0. None of the regimens utilized, either live, or formalin-inactivated, resulted in full survival. Elevated levels of IgA were detected in mice primed specifically with live or inactivated L. rhamnossus; however, the present invention has since shown that protection elicited by Lactobacillus-priming is fully antibody-independent [Percopo et al., 2014a Methods Mol. Biol. 1178: 257-266]. Also, formalin is an inadvisable preservative given the experience with this additive and RSV vaccines [Anderson, 2013 Semin. Immunol. 25: 160-171]. Hori and colleagues [2001 Clin. Diag. Lab. Immunol. 8: 593-597] found that administration of heat-inactivated L. casei strain Shirota, three inoculations (10 mg/mL), once per day prior to virus challenge, protected the lower respiratory tract from Influenza A/PR/8/34 inoculated into the upper respiratory tract, and subsequently eluted down via PBS washes, although protection was not absolute (70% was presented). Similarly, Harata and colleagues [2010. Lett. Appl. Microbiol. 50: 597-602] utilized the same protocol as Hori et al., [2001 Clin. Diag. Lab. Immunol. 8: 593-597] although with heat-inactivated L. rhamnossus GG prior to virus challenge. Nearly identical results were obtained (60% survival in response to Lactobacillus vs. 15% survival without). No other intervals or regimens were evaluated. The authors evaluated cytokine responses to L. rhamnossus challenge, but did not examine specific suppression in primed mice in response to virus challenge. Similar results were obtained by Izumo and colleagues [2010 Int. Immunopharmacol. 10: 1101-1106] using this protocol in a study featuring L. pentosus S-PT84.

Tomosada and colleagues [2013 BMC Immunology 14: 40] examined L. rhamnossus CRL1505 and CRL1506 in a study of BALB/c challenge with human RSV. RSV is a human pathogen, and does not replicate or elicit disease-related pathology in the BALB/c mouse. The results from this study cannot be compared to those utilizing replication-competent virus pathogens that elicit disease pathology in rodents such as PVM or mouse-passaged Influenza A strains.

It may be deduced from published work in mouse model systems that administration of live or heat-inactivated cells of probiotic Lactobacillus directly to the respiratory mucosa can benefit the host by protecting against the lethal sequelae of acute respiratory infection. In order to develop these observations into an effective therapeutic for the prevention, treatment, and/or the relief of symptoms associated with acute respiratory tract infections, the relationship between Lactobacillus administration and the suppression of virus-induced inflammation is to be clarified, a major determinant of the severity of disease. The minimum effective and maximum tolerated doses, both in terms of number of cells and dosing intervals, as well as timing with respect to virus exposure (as possible) is to be determined.

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

It is to be understood that the terminology used herein is for the purpose of describing particular embodiments only and is not intended to limit the scope of the present invention. In this specification and in the claims that follow, reference will be made to a number of terms that shall be defined to have the following meanings.

As used herein, the term “treating” means ameliorating, attenuating, mitigating, reducing, improving, remedying or any variation of these terms, when used in the claims and/or the specification includes any measurable decrease in disease, disorder, or condition through some action.

The term “preventing” means to stop, hinder, or to provide any measurable decrease or complete inhibition of the onset of symptoms or magnitude of severity of a disease, disorder, or condition.

The terms “therapeutically effective amount” refer to an amount or dosage of a composition of the invention at high enough levels to improve the condition to be prevented and/or treated, but low enough to avoid serious side effects (at a reasonable benefit/risk ratio), within the scope of sound medical judgment. The therapeutically effective amount or dosage of a composition of the invention may vary with the particular condition being treated, the age and physical condition of the patient being treated, the severity of the condition, the duration of treatment, the nature of concurrent therapy, the specific form of the source employed, and the particular vehicle from which the composition is applied.

“Patient”, “host”, or “subject” refers to mammals and includes humans and non-human mammals.

“Treating” or “treatment” of a disease, disorder, condition or symptom in a patient refers to 1) preventing the disease, disorder, condition or symptom from occurring in a patient that is predisposed or does not yet display symptoms of the disease; 2) inhibiting the disease, disorder or symptom or arresting its development; or 3) ameliorating or causing regression of the disease, disorder, or symptom associated with the disease.

As used herein, the term “immune response” includes all of the specific and non-specific processes and mechanisms involved in how the body defends, tolerates, and repairs itself against bacteria, viruses, fungi, parasites, allergens and all substances, insults, challenges, biological and/or physical invasions of the body that are harmful to the body.

As used herein “enhancing immune response” means promoting a functional change to the immune system or its response which provides a benefit to the mammal. “Enhancing” the immune response also includes prevention, treatment, cure, mitigation, amelioration, inhibition and/or alleviation of a respiratory condition and/or the relief of symptoms as a result of a respiratory condition.

As used herein, a “probiotic” microorganism or strain of microorganism confers beneficial functions and/or effects on a host animal when administered at a therapeutically effective amount. As used herein “immunobiotic” microorganisms include bacteria, bacterial homogenates, ground bacterial cells, bacterial proteins, bacterial extracts, bacterial ferment supernatants, and mixtures thereof that have positive impact on the immune and/or inflammatory response of the host, leading to beneficial effects on health and well-being. Immunobiotic microorganisms also include natural and/or genetically modified microorganisms, viable or dead; processed compositions of microorganisms; their constituents and components such as proteins and carbohydrates, extracts, distillates, isolates, purified fractions, and mixtures thereof of bacterial ferments that have a beneficial impact on a host. Although a use of immunobiotic microorganisms herein can be in the form of viable cells, use can be extended to non-viable cells such as inactivated cultures, or compositions containing beneficial factors expressed by the immunobiotic microorganisms. Inactivated cultures may include thermally-killed microorganisms, or microorganisms killed by exposure to UV, altered pH or subjected to pressure. The term “immunobiotic” microorganisms is further intended to include metabolites generated by the microorganisms during fermentation, if such metabolites are not separately indicated. These metabolites may be released to the medium during fermentation, or they may be stored within the microorganism and released via mechanical or biochemical processes as part of the inactivation process.

The abbreviation CFU or cfu (referring to “colony-forming unit”) as used herein designates the number of bacterial cells revealed by microbiological counts on agar plates, as will be commonly understood in the art. CFU will also refer to inactivated organisms, wherein the microbiological counts will have been determined prior to inactivation.

The term “cells” when used to describe inoculum dose “cells/mL” refer to CFU or cfu equivalent as whole cells or mixture of whole and lysed cells that may result from the inactivation process.

The term “pharmaceutically acceptable carrier” refers to any solid, liquid or gas combined with components of the compositions of the present invention to deliver the components to the user. These vehicles are generally regarded as safe for use in humans, and are also known as carriers or carrier systems.

The present invention provides for novel products, methods and uses for preventing and/or the treating an inflammatory disease, disorder, condition, symptoms and/or pathology thereof. In further embodiments, the present invention provides immunobiotics for these purposes.

For example, the present invention provides means for preventing and/or treating the inflammatory symptoms and/or pathology associated with respiratory infections.

Also provided is a pharmaceutical composition comprising a pharmaceutically acceptable carrier or excipient and a therapeutically effective amount of one or more Lactobacillus strains.

Also provided are methods for preparing such Lactobacillus compositions and for their therapeutic uses.

One embodiment of the invention provides for the treatment and/or prevention of respiratory infections in normal, healthy subjects.

Another embodiment of the invention provides for the treatment and/or prevention of the pathology and/or symptoms associated with respiratory infections in normal, healthy subjects

Another embodiment of the invention provides a method of limiting virus replication in previously normal, healthy subjects.

In another embodiment, the invention provides for treatment and/or prevention of inflammatory responses, conditions, pathology and/or symptoms associated with respiratory infections, and in particular, from viral respiratory infections in subjects having an increased susceptibility and/or adverse reaction to respiratory infections as well as in previously normal, healthy subjects. In both embodiments, the method consists of administering a composition comprising one or more isolated, non-pathogenic, Lactobacillus species or strains directly to the upper and/or lower respiratory tract of the subject.

Subjects have an increased susceptibility and/or adverse reaction to respiratory infection when they are more likely than a normal, healthy host to acquire and/or have an adverse reaction to a respiratory infection. Such hosts may have, for example, asthma, cystic fibrosis, chronic obstructive pulmonary disorder, allergic rhinitis, nasal polyps and acute respiratory distress syndrome.

The methods of the present invention comprise administering a composition comprising one or more isolated, Lactobacillus species or strains to the upper and/or lower respiratory tract of the subject or host.

In further embodiments, the immunobiotic used in the compositions of the present invention is a single species, or a mixture of species, of a probiotic microorganism. Even more preferred are microorganisms which are probiotic bacteria. Further preferred are probiotic bacteria which can alter the immune/inflammatory response, so that the host can survive from an otherwise lethal respiratory virus infection. The probiotic bacteria may advantageously be selected from any previously known or newly discovered strain of Lactobacillus, or parts thereof which are capable of inducing a beneficial response from the host. Lactobacillus, or parts thereof, which are capable of altering the immune response as indicated above, may be used. Similarly, immunobiotic bacteria may be used as a whole cell preparation either live or as an inactivated preparation, as long as they are capable of having a positive impact on the immune and/or inflammatory response of the host, leading to a beneficial effect on health and well-being.

In still further embodiments, the immunobiotic bacteria used in the compositions of the present invention is a single species consisting of Lactobacillus plantarum strains suitable for use herein include ATCC 10241, ATCC 14431, ATCC 39268, ATCC 21028, ATCC 55324, ATCC 39542, ATCC 14917, ATCC 700211, ATCC BAA-793, ATCC 4008, ATCC 8014, ATCC 10012, ATCC 49445, ATCC 53187, ATCC 700210, ATCC BAA-171, DSMZ 10492, DSMZ 1055, DSMZ 12028, DSMZ 24624, DSMZ 2648, DSMZ 6872 and DSMZ 16365.

In still further embodiments, the immunobiotic bacteria used in the compositions of the present invention is a single species consisting of whole cell, heat-inactivated Lactobacillus plantarum (ATCC BAA-793).

In still further embodiments, the immunobiotic bacteria used in the compositions of the present invention is a single species consisting of whole cell, heat-inactivated Lactobacillus plantarum (ATCC BAA-793) which is delivered directly to the upper and/or lower respiratory tract

In still a further embodiments, the immunobiotic bacteria used in the compositions of the present invention is a single species consisting of whole cell, heat-inactivated Lactobacillus plantarum (ATCC BAA-793) which is delivered directly to the upper respiratory tract as a dry powder.

In still a further embodiments, the immunobiotic bacteria used in the compositions of the present invention is a single species consisting of whole cell, heat-inactivated Lactobacillus plantarum (ATCC BAA-793) is delivered directly to the upper respiratory tract as a dry powder using an intranasal delivery device.

Non-limiting examples of Lactobacillus suitable for use herein include one or more species of that are selected from the group consisting of L. acetotolerans, L. acidifarinae, L. acidipiscis, L. acidophilus, L. agilis, L. algidus, L. alimentarius, L. amylolyticus, L. amylophilus, L. amylotrophicus, L. amylovorus, L. animalis, L. antri, L. apodemi, L. aviaries, L. bifidus L. bifermentans, L. brevis, L. buchneri, L. bulgaricus, L. camelliae, L. casei, L. catenaformis, L. ceti, L. coleohominis, L. collinoides, L. composti, L. concavus, L. coryniformis, L. crispatus, L. crustorum, L. curvatus, L. delbrueckii subsp. Delbrueckii, L. delbrueckii subsp. Bulgaricus, L. delbrueckii subsp. Lactis, L. dextrinicus, L. diolivorans, L. equi, L. equigenerosi, L. farraginis, L. farciminis, L. fermentii, L. fermentum, L. fornicalis, L. fructivorans, L. frumenti, L. fuchuensis, L. gallinarum, L. gasseri, L. gastricus, L. ghanensis, L. graminis, L. hammesii, L. hamster, L. harbinensis, L. hayakitensis, L. helveticus, L. hilgardii, L. homohiochii, L. iners, L. ingluviei, L. intestinalis, L. jensenii, L. johnsonii, L. kalixensis, L. kefiranofaciens, L. kefiri, L. kimchii, L. kitasatonis, L. kunkeei, L. lactis, L. leichmannii, L. lindneri, L. malefermentans, L. mali, L. manihotivorans, L. mindensis, L. mucosae, L. murinus, L. nagelii, L. namurensis, L. nantensis, L. oligofermentans, L. oris, L. panis, L. pantheris, L. parabrevis, L. parabuchneri, L. paracasei, L. paracollinoides, L. parafarraginis, L. parakefiri, L. paralimentarius, L. paraplantarum, L. pentosus, L. perolens, L. plantarum, L. pontis, L. psittaci, L. rennin, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. thailandensis, L. thermophilus, L. ultunensis, L. vaccinostercus, L. vaginalis, L. versmoldensis, L. vini, L. vitulinus, L. zeae, and L. zymae.

Other non-limiting examples of Lactobacillus strains suitable for use herein include the Lactobacillus acidophilus strain identified as CL-92 deposited in Japan at International Patent Organism Depository, FERM BP-4981, the Lactobacillus acidophilus strain identified as CL0062 deposited in Japan at International Patent Organism Depository, FERM BP4980, and the Lactobacillus fermentum strain identified as CP34 and deposited in Japan at International Patent Organism Depository, FERM BP-8383. These organisms, have been shown, as described in US Patent Application Publication Number US 2005/0214270.

Other non-limiting examples of Lactobacillus strains suitable for use herein include Lactobacillus rhamnosus DSM 16605 (DSMZ—Deutsche Sammlung von Mikroorganismen and Zellkulturen GmbH, Braunsweig-Germany, on 20 Jul. 2004; depositor Anidral S.r. L.); Lactobacillus plantarum LMG P-21021 (BCCM LMG—Belgian Coordinated Collections of Micro-organisms, Universiteit Gent, on 16 Oct. 2001, depositor Mofin S.r. L.); Lactobacillus plantarum LMG P-21020 (BCCM LMG—Belgian Coordinated Collections of Micro-organisms, Universiteit Gent, on 16 Oct. 2001, depositor Mofin S.r. L.); Lactobacillus plantarum LMG P-21022 (BCCM LMG—Belgian Coordinated Collections of Micro-organisms, Universiteit Gent, on 16 Oct. 2001, depositor Mofin S.r. L.); Lactobacillus plantarum LMG P-21023 (BCCM LMG—Belgian Coordinated Collections of Micro-organisms, Universiteit Gent, on 16 Oct. 2001, depositor Mofin S.r. L.).

The bacteria of the invention can be administered in the form of viable bacteria or non-viable bacteria such as killed or inactivated cultures. Killed cultures can include thermally killed bacteria, or bacteria killed by exposure to UV, altered pH, subjected to pressure, or subject to other methods of killing or inactivating.

In one embodiment of the invention, the bacteria of the invention can be viable or not viable.

Compositions of the invention can be administered at any dose between 1×10³ to 1×10¹³ CFU (or CFU equivalent, after inactivation) Lactobacillus. Any number of doses can be administered per day, per week, per month, per year, or per multiple years.

Bacteria used according to the invention may be obtained by any available means. A variety of bacterial species and strains are commercially available or available from American Type Culture Collection Catalogue (Manassas, Va.). Bacteria may also be cultured, for example, in liquid or on solid media, following routine and established protocols and isolated from the medium by any available means, such as centrifugation or filtration from liquid medium or mechanical removal from solid medium, for example. Exemplary methods are described in Methods in Cloning Vol. 3, eds. Sambrook and Russell, Cold Spring Harbor Laboratory Press (2001) and references cited within. In certain embodiments, one or more of the bacteria included in the composition are isolated or separated from its growth medium by centrifugation. Methods of isolating bacteria from medium are well-known and available in the art.

The present invention is directed to compositions and pharmaceutical compositions that have utility as novel treatments, the relief of symptoms, and/or preventative therapies for inflammatory disease, conditions or pathology.

The present invention is directed to compositions and pharmaceutical compositions that have utility as novel treatments and/or preventative therapies where the inflammatory disease, conditions or pathology and/or symptoms are due to respiratory infections, and in particular, from respiratory virus infections.

In one embodiment, the present invention is directed to novel Lactobacillus treatments and/or preventative therapies or the relief of symptoms associated with viral infections located in the subject's upper respiratory tract.

In other embodiments, the present invention is directed to novel Lactobacillus treatments and/or preventative therapies and/or the relief of symptoms in a subject associated with viral infections located in the subject's lower respiratory tract.

In still other embodiments, the present invention is directed to novel Lactobacillus treatments and/or preventative therapies and/or the relief of symptoms in a subject for viral infections in the subject that are selected from the virus Families including Picornoviridae, Paramyxoviridae, Orthomyxoviridae, Coronaviridae, and Adenoviridae.

Viruses are classified by evaluating several characteristics, including the type of viral genome. Viral genomes can be comprised of DNA or RNA, can be double-stranded or single-stranded (which can further be positive-sense or negative-sense), and can vary greatly by size and genomic organization. An RNA virus is a virus that has RNA (ribonucleic acid) as its genetic material. Infectious RNA virus usually consists of single-stranded RNA (ssRNA). RNA viruses can be further classified according to the sense or polarity of their RNA into negative-sense and positive-sense. Positive-sense viral RNA is similar to mRNA and thus can be immediately translated by the host cell. Negative-sense viral RNA is complementary to mRNA and thus must be converted to positive-sense RNA by an RNA polymerase before translation.

Single-stranded RNA viruses make up a large superfamily of viruses from many distinct subfamilies. These viruses cause pathologies ranging from mild phenotypes to severe debilitating disease. The composition of a single strand RNA virus includes, at least, the following families: levi-, narna-, picorna-, dicistro-, marna-, sequi-, como-, poty-, calici-, astro-, noda-, tetra-, luteo-, tombus-, corona-, arteri-, roni-, flavi-, toga-, bromo-, tymo-, clostero-, flexi-, seco-, barna, ifla-, sadwa-, chera-, hepe-, sobemo-, umbra-, tobamo-, tobra-, hordei-, furo-, pomo-, peclu-, beny-, ourmia-, influenza-, rhino- and idaeovirus.

In one embodiment of the present invention, the compositions described herein are useful for preventing or treating viral infections and/or symptoms thereof in a subject caused by a negative-sense or positive-sense single-stranded RNA virus.

In certain embodiments, the present invention is directed to novel Lactobacillus-based treatments and/or preventative therapies in a subject for viral infections and/or symptoms thereof that are selected from the group consisting of rhinovirus, influenza virus, coronavirus, parainfluenza virus, adenovirus, enterovirus, respiratory syncytial virus, SARS, MERS, metapneumovirus, and paramyxovirus.

Another embodiment of the present invention provides a method of treating a virus infection and/or symptoms thereof in a subject suffering from the virus infection comprising administering to the subject's respiratory tract a composition comprising one or more strains of Lactobacillus bacteria.

Another embodiment of the present invention provides a method of treating a virus infection and/or symptoms thereof in a subject suffering from the virus infection comprising administering to the subject's respiratory tract a composition comprising of whole cell, heat-inactivated Lactobacillus plantarum ATCC BAA-793.

Another embodiment of the present invention provides a method of preventing a virus infection and/or the relief of symptoms associated with viral infection in a subject comprising administering to the subject's lower respiratory tract a composition comprising one or more strains of Lactobacillus bacteria.

Another embodiment of the present invention provides a method of preventing a virus infection and/or symptoms thereof in a subject comprising administering to the subject's lower respiratory tract a composition comprising of whole cell, heat-inactivated Lactobacillus plantarum ATCC BAA-793.

Another embodiment of the present invention provides a method of preventing a virus infection and/or the relief of symptoms associated with viral infection in a subject comprising administering to the subject's upper respiratory tract a composition comprising one or more strains of Lactobacillus bacteria.

Another embodiment of the present invention provides a method of preventing a virus infection and/or symptoms thereof in a subject comprising administering to the subject's upper respiratory tract a composition comprising of whole cell, heat-inactivated Lactobacillus plantarum ATCC BAA-793.

Another embodiment of the present invention provides a method of treating rhinovirus, respiratory syncytial virus, and/or influenza virus, parainfluenza, metapneumovirus, and adenovirus, infection and/or the relief of symptoms associated with these viruses in a subject suffering from rhinovirus and/or influenza virus infection comprising administering to the subject's lower respiratory tract a composition comprising one or more strains of Lactobacillus bacteria.

Another embodiment of the present invention provides a method of treating a rhinovirus, respiratory syncytial virus, and/or influenza virus, parainfluenza, metapneumovirus, and adenovirus infection and/or the relief of symptoms associated with these viruses in a subject suffering from the rhinovirus, respiratory syncytial virus and/or influenza virus infection comprising administering to the subject's lower respiratory tract a composition comprising of whole cell, heat-inactivated Lactobacillus plantarum ATCC BAA-793.

Another embodiment of the present invention provides a method of treating a rhinovirus, respiratory syncytial virus, and/or influenza virus, parainfluenza, metapneumovirus, and adenovirus infection and/or the relief of symptoms associated with these viruses in a subject suffering from the rhinovirus, respiratory syncytial virus and/or influenza virus infection, respectively, comprising administering to the subject's upper respiratory tract a composition comprising one or more strains of Lactobacillus bacteria.

Another embodiment of the present invention provides a method of treating a rhinovirus, respiratory syncytial virus, and/or influenza virus, parainfluenza, metapneumovirus, and adenovirus infection and/or the relief of symptoms associated with these viruses in a subject suffering from the rhinovirus, respiratory syncytial virus, and/or influenza virus, parainfluenza, metapneumovirus, and adenovirus infection, respectively, comprising administering to the subject's upper respiratory tract a composition comprising of whole cell, heat-inactivated Lactobacillus plantarum ATCC BAA-793.

In other embodiments, the compositions described herein are useful for preventing or treating viral infections and/or the relief of symptoms associated viral infections in a subject where the infection is caused by a virus belonging to the following families: levi-, narna-, picorna-, dicistro-, mama-, sequi-, como-, poty-, calici-, astro-, noda-, tetra-, luteo-, tombus-, corona-, arteri-, roni-, flavi-, toga-, bromo-, tymo-, clostero-, flexi-, seco-, barna, ifla-, sadwa-, chera-, hepe-, sobemo-, umbra-, tobamo-, tobra-, hordei-, furo-, pomo-, peclu-, beny-, ourmia-, and idaeovirus.

Compositions, methods and pharmaceutical compositions for treating viral infections and/or the relief of symptoms associated viral infections in a subject's respiratory tract, by administering to the subject having a viral infection a composition comprising one or more strains of Lactobacillus bacteria, are disclosed. Methods for preparing such compositions and methods of using the compositions and pharmaceutical compositions thereof are also disclosed. In particular, the treatment and prophylaxis of viral infections and/or symptoms thereof such as those caused by RNA or DNA viruses are disclosed.

In other embodiments, the compositions described herein are useful for treating viral infections and/or the relief of symptoms associated viral infections in a subject where the infection is caused by any one or more viruses selected from the group consisting of, rhinovirus (A-C), coxsackievirus, influenza A virus, influenza B virus, adenovirus, metapneumovirus, parainfluenzavirus, coronavirus, Severe Acute Respiratory Syndrome (SARS), Middle East Respiratory Syndrome (MERS), respiratory syncytial virus (RSV), enterovirus, and avian and/or swine influenza virus.

In other embodiments, the compounds described herein are useful for treating viral infections and/or the relief of symptoms associated viral infections in a subject where the infection is caused by any of the human enteroviruses A-D.

In other embodiments, the compounds described herein are useful for treating viral infections and/or the relief of symptoms associated viral infections in a subject where the infection is caused by enterovirus A71.

In other embodiments, the compounds described herein are useful for treating viral infections and/or the relief of symptoms associated viral infections in a subject where the infection is caused by any of the human rhinoviruses A-C.

In other embodiments, the compounds described herein are useful for treating viral infections and/or the relief of symptoms associated viral infections in a subject where the infection is caused by human rhinovirus A.

In other embodiments, the compounds described herein are useful for treating viral infections and/or the relief of symptoms associated viral infections in a subject where the infection is caused by human rhinovirus B.

In other embodiments, the compounds described herein are useful for treating viral infections and/or the relief of symptoms associated viral infections in a subject where the infection is caused by human rhinovirus C.

In one embodiment of the present invention, the compositions described herein are useful for preventing or treating viral infections and/or the relief of symptoms associated viral infections in a subject caused by a DNA virus.

The Lactobacillus compositions of the present invention may conveniently be administered by any inhaled route. The compositions herein may be administered in conventional dosage forms, such as from an inhaler device and can be prepared by combining a Lactobacillus composition with standard pharmaceutical carriers according to conventional procedures. For example, nasal drops can be instilled in the nasal cavity by tilting the head back sufficiently and apply the drops into the nares. The drops may also be inhaled through the nose. Alternatively, a liquid preparation may be placed into an appropriate device so that it may be aerosolized for inhalation through the nasal cavity. For example, the therapeutic agent may be placed into a plastic bottle atomizer. In one embodiment, the atomizer is advantageously configured to allow a substantial amount of the spray to be directed to the upper one-third region or portion of the nasal cavity. Alternatively, the spray is administered from the atomizer in such a way as to allow a substantial amount of the spray to pass the nasal valve and to be directed to the upper one-third region or portion of the nasal cavity. By “substantial amount of the spray” it is meant herein that at least about 50%, further at least about 70%, but preferably at least about 80% or more of the spray passes the nasal valve and is directed to the upper and distal portion of the nasal cavity with about 10% or more reaching the upper third of the nasal cavity. Administered spray and drops can be a single dose or multiple doses.

These procedures may involve mixing, granulating and compressing or dissolving the ingredients as appropriate to the desired preparation. It will be appreciated that the form and character of the pharmaceutically acceptable diluent is dictated by the amount of Lactobacillus active ingredient with which it is to be combined, the route of administration and other well-known variables. The carrier(s) must be “acceptable” in the sense of being compatible with the other ingredients of the formulation and not deleterious to the recipient thereof.

The Lactobacillus compositions of the present invention, may also be administered by inhalation; that is by intranasal and oral inhalation administration. Appropriate dosage forms for such administration, such as an aerosol formulation or a metered dose inhaler, may be prepared by conventional techniques. In one embodiment of the present invention, the agents of the present invention are delivered via oral inhalation or intranasal administration. Appropriate dosage forms for such administration, such as an aerosol formulation or a metered dose inhaler, may be prepared by conventional techniques.

For administration by inhalation the compositions may be delivered in the form of an aerosol spray presentation from pressurized packs or a nebulizer, with the use of a suitable propellant, e.g. dichlorodifluoromethane, trichlorofluoromethane, dichlorotetrafluoroethane, a hydrofluoroalkane such as tetrafluoroethane or heptafluoropropane, carbon dioxide or other suitable gas. In the case of a pressurized aerosol the dosage unit may be determined by providing a valve to deliver a metered amount. Capsules and cartridges of gelatin for use in an inhaler or insufflator may be formulated containing a powder mix of a Lactobacillus composition of the invention and a suitable powder base such as trehalose, lactose or starch.

Dry powder compositions for topical delivery to the lung by inhalation may, for example, be presented in capsules and cartridges of for example HPMC, gelatin or blisters of for example laminated aluminum foil, for use in an inhaler or insufflator. Powder blend formulations generally contain a powder mix for inhalation of the compositions of the invention and a suitable powder base (carrier/diluent/excipient substance) such as mono-, di or poly-saccharides (e.g., trehalose, lactose or starch).

Each capsule or cartridge may generally contain between 20 μg-50 mg of the Lactobacillus compositions of the present invention. Alternatively, the compositions of the invention may be presented without excipients. Suitably, the packing/medicament dispenser is of a type selected from the group consisting of a reservoir dry powder inhaler (RDPI), a multi-dose dry powder inhaler (MDPI), and a metered dose inhaler (MDI). By reservoir dry powder inhaler (RDPI) it is meant an inhaler having a reservoir form pack suitable for comprising multiple (un-metered doses) of medicament (e.g., Lactobacillus compostions) in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the patient for inhalation. By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In a preferred aspect, the carrier has a blister pack form, but it could also, for example, comprise a capsule-based pack form or a carrier onto which medicament has been applied by any suitable process including printing, painting and vacuum occlusion.

In the case of multi-dose delivery, the formulation can be pre-metered (e.g. as in Diskus, see U.S. Pat. Nos. 6,632,666, 5,860,419, 5,873,360 5,622,166 and 5,590,645 or Diskhaler, see, U.S. Pat. Nos. 4,627,432, 4,778,054, 4,811,731, 5,035,237, the disclosures of which are hereby incorporated by reference) or metered in use (e. g. as in Turbuhaler, see U.S. Pat. No. 4,524,769 or in the devices described in U.S. Pat. No. 6,321,747 the disclosures of which are hereby incorporated by reference). An example of a unit-dose device is Rotahaler (see U.S. Pat. Nos. 4,353,656 and 5,724,959, the disclosures of which are hereby incorporated by reference).

The Diskus inhalation device comprises an elongate strip formed from a base sheet having a plurality of recesses spaced along its length and a lid sheet hermetically but peelably sealed thereto to define a plurality of containers, each container having therein an inhalable formulation containing a composition of the present invention preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet. In one aspect, the multi-dose pack is a blister pack comprising multiple blisters for containment of medicament in dry powder form. The blisters are typically arranged in regular fashion for ease of release of medicament there from. In one aspect, the multi-dose blister pack comprises plural blisters arranged in generally circular fashion on a disc-form blister pack. In another aspect, the multidose blister pack is elongate in form, for example comprising a strip or a tape. In one aspect, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peel-able members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary. Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable. The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined there between and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085. Additionally, intra-nasal delivery of the present compounds is effective. A suitable intra-nasal delivery device would be the unit dose system (UDS) from Aptar Pharma which is a single shot delivery device applicable for therapies where a small and very precise amount of active drug formulation is required in a single nasal or sub-lingual shot. The UDS device is capable of delivering a powder dosage, with maximum filling volume 140 mm³, while protecting the drug product.

To formulate an effective Lactobacillus nasal composition, preferably the medicament is delivered readily to all portions of the nasal cavities (the target tissues) where it performs its pharmacological function. Additionally, preferably the medicament remains in contact with the target tissues for relatively long periods of time. The longer the medicament remains in contact with the target tissues, the medicament preferably is capable of resisting those forces in the nasal passages that function to remove particles from the nose. Such forces, referred to as ‘mucociliary clearance’, are recognized as being extremely effective in removing particles from the nose in a rapid manner, for example, within 10-30 minutes from the time the particles enter the nose.

Other desired characteristics of a nasal composition are that it preferably does not contain ingredients which cause the user discomfort, that it has satisfactory stability and shelf-life properties, and that it does not include constituents that are considered to be detrimental to the environment, for example ozone depletors. A suitable dosing regimen for the formulation of the present invention when administered to the nose would be for the patient to inhale deeply subsequent to the nasal cavity being cleared. During inhalation, the formulation would be applied to one nostril while the other is manually compressed. This procedure would then be repeated for the other nostril. One means for applying the formulation of the present invention to the nasal passages is by use of a pre-compression pump. Most preferably, the pre-compression pump will be a VP7 model manufactured by Valois SA. Such a pump is beneficial as it will ensure that the formulation is not released until a sufficient force has been applied, otherwise smaller doses may be applied. Another advantage of the precompression pump is that atomization of the spray is ensured as it will not release the formulation until the threshold pressure for effectively atomizing the spray has been achieved. Typically, the VP7 model may be used with a bottle capable of holding 10-50 ml of a formulation. Each spray will typically deliver 50-100 μl of such a formulation; therefore, the VP7 model is capable of providing at least 100 metered doses.

Spray compositions for topical delivery to the lung by inhalation may for example be formulated as aqueous solutions or suspensions or as aerosols delivered from pressurized packs, such as a metered dose inhaler, with the use of a suitable liquefied propellant. Aerosol compositions suitable for inhalation can be either a suspension or a solution and generally contain the compositions of the present invention and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetra-fluoroethane, especially 1,1, 1,2-tetrafluoroethane, 1,1, 1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants, e.g., oleic acid or lecithin and cosolvents, e.g. ethanol. Pressurized formulations will generally be retained in a canister (e.g. an aluminum canister) closed with a valve (e.g. a metering valve) and fitted into an actuator provided with a mouthpiece. Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient as produced may be size reduced by conventional means e.g., by micronization. The desired fraction may be separated out by air classification or sieving. Suitably, the particles will be crystalline in form. When an excipient such as lactose is employed, generally, the particle size of the excipient will be much greater than the inhaled medicament within the present invention. When the excipient is lactose it will typically be present as milled lactose, wherein not more than 85% of lactose particles will have a MMD of 60-90 μm and not less than 15% will have a MMD of less than 15 μm. Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulization may be formulated with an aqueous vehicle with the addition of agents such as acid or alkali, buffer salts, isotonicity adjusting agents or antimicrobials. They may be sterilized by filtration or heating in an autoclave, or presented as a non-sterile product. Suitably, administration by inhalation may preferably target the organ of interest for respiratory diseases, i.e. the lung, and in doing so may reduce the efficacious dose needed to be delivered to the patient. In addition, administration by inhalation may reduce the systemic exposure of the compound thus avoiding effects of the compound outside the lung.

PVM is a natural rodent pathogen that is in the same Family (Paramyxovirdae) and genus (Pneumovirus) as the common human pediatric pathogen, respiratory syncytial virus (RSV). However, unlike RSV, PVM undergoes robust replication in mouse lung tissue, and generates clinical findings and pathophysiology of a severe model of viral infection in a rodent host [Bem et al., 2011 Am J Physiol Lung Cell Mol Physiol. 301:L148-L156; Rosenberg & Domachowske, 2012 Curr Med Chem 19: 1424-1431]. PVM infection induces a massive inflammatory response that correlates with lethal pathology and as such is an informative experimental model in which to evaluate responses to a targeted anti-inflammatory therapeutic agent. RSV cannot be studied in this manner.

Aspects of the present invention may also be directed to methods of treating at least one symptom of a cold or flu comprising administering to the subject a composition comprising one or more species of Lactobacillus bacteria. At least one symptom of a cold or flu may be selected from the group consisting of stuffy nose, runny nose, coughing, aches, pains, sore throat, fever, chest congestion sinus pain, and sinus pressure. In certain embodiments, the composition comprising one or more species of Lactobacillus bacteria is administered after the at least one symptom of a cold or flu has been experience by a subject. In certain embodiments, the one or more species of Lactobacillus bacteria may be administered to the intranasal mucosa of a subject. Upon administration of the one or more species of Lactobacillus bacteria the severity of the at least one symptom of a cold or flu may be lessened. Upon administration of the one or more species of Lactobacillus bacteria the duration of the at least one symptom of a cold or flu may be lessened.

Additional aspects of the present invention may be directed to methods of preventing at least one symptom of a cold or flu comprising administering to the subject a composition comprising one or more species of Lactobacillus bacteria, wherein the at least one symptom of a cold or flu is selected from the group consisting of stuffy nose, runny nose, coughing, aches, pains, sore throat, fever, chest congestion sinus pain, and sinus pressure.

Further aspects of the present invention may be directed to methods of ameliorating at least one symptom of a cold or flu comprising administering to the subject a composition comprising one or more species of Lactobacillus bacteria, wherein the at least one symptom of a cold or flu is selected from the group consisting of stuffy nose, runny nose, coughing, aches, pains, sore throat, fever, chest congestion sinus pain, and sinus pressure.

### Materials and Methods

Generation of Infection with PVM Strain J3666:

All experiments with PVM were performed with pneumonia virus of mice (PVM) strain J3666. This strain has been maintained in mice and not passaged in tissue culture. Lungs from virus infected mice were homogenized in tissue culture medium (IMDM with 10% fetal calf serum+2 mM glutamine+pen/strep, 1 mL per mouse). Clarified medium was snap frozen in aliquots, stored in liquid nitrogen, and defrosted just prior to use. Our stocks of mouse-passaged PVM have been measured at 10⁵ TCID₅₀ units/mL as described [Percopo et al., Methods Mol. Biol. Chapter 22, 1178: 257-266]. PVM stocks were prepared in and diluted in tissue culture medium (IMDM with 10% FCS, 2 mM glutamine with pen/strep) as vehicle for inoculation unless otherwise specified. BALB/c mice under isoflurane anaesthesia receive 50 microliters of virus diluted at 1:10,000; C57BL/6 mice under isoflurane anaesthesia receive 50 microliters of virus diluted 1:1000. Anaesthetized mice were held in a supine position with neck hyperextended and receive the 50 microliter dose in 5-6 small aliquots. Once inoculated, mice were returned to their cages in prone position and permitted to awaken/recover from anaesthesia.

### Influenza A/HK/68 (H3N2):

Egg-passaged virus was used to inoculate BALB/c mice; mouse lungs were washed in cold PBS and homogenized in cold PBS with pen/strep (1-2 mL/mouse). Clarified supernatants were snap frozen and stored at −80° C. Virus stocks were defrosted just prior to use and used at a 1:50 dilution to inoculate BALB/c mice, 2.5 microliter per nare (5 microliter per mouse). Anaesthetized mice were held in a supine position with neck hyperextended during the inoculation and returned to their cages in prone position and permitted to awaken/recover from anaesthesia.

### Preparation of Live Lactobacillus, (Lp-F00).

L. plantarum ATCC BAA-793 (ATCC BAA-793) from frozen stock is grown overnight in 50 mL MRS medium at 37° C. with rotary shaking at 250 rpm. Colony forming units (CFU)/mL was determined from the OD-600. Bacteria are harvested by centrifugation, washed once with PBS and resuspended in PBS at 2×10¹⁰ CFU/mL as described in the data supplement to Gabryszewski and colleagues [2011 J. Immunol. 186: 1151-1161].

### Preparation of Heat-Inactivated Lactobacillus, (Lp-F0).

L. plantarum ATCC BAA-793 (ATCC BAA-793) from frozen stock was grown overnight in 50 mL MRS medium at 37° C. with rotary shaking at 250 rpm. Colony forming units (CFU)/mL was determined from the OD-600. Bacteria are harvested by centrifugation, washed once with PBS and resuspended in PBS at ˜2×10¹¹ CFU/mL. Bacteria were heated to 95° C. for 10 minutes, then snap frozen on dry ice. This was repeated 3 times. After final defrost, bacteria were combined, diluted to 10¹¹/mL in PBS with 0.1% bovine serum albumin, and frozen at 10¹¹ cells/mL as described in Gabryszewski and colleagues [2011 J. Immunol. 186: 1151-1161].

Preparation of Lactobacillus plantarum Formulation 1, (Lp-F1).

Lactobacillus plantarum (ATCC BAA-793; ATCC BAA-793) was grown in Soytone-MRS+5% glucose to an OD₆₀₀ of 21. Samples were withdrawn from the fermenter, and colony forming units (CFU) per mL measured. Immediately after sampling, the fermenter was heated to 60° C. and held for 30 minutes. The cells were harvested by centrifugation and re-suspended at 1E10 cells/mL in sterile 1×PBS+20% glycerol. Inactivation was confirmed by 48 hour incubation in Soytone-MRS broth and agar plates.

Preparation of Lactobacillus plantarum Formulation 2, (Lp-F2).

Lactobacillus plantarum (ATCC BAA-793; ATCC BAA-793) was grown in Soytone-MRS+5% glucose to an OD₆₀₀ of 21. Samples were withdrawn from the fermenter, and colony forming units (CFU) per mL measured. The cells were harvested by centrifugation, re-suspended at 1×10¹¹ cell/mL in sterile 1×PBS+20% glycerol, and placed in a water bath pre-equilibrated to 70° C. for 30 minutes. Inactivation was confirmed by 48 hour incubation in Soytone-MRS broth and agar plates.

Preparation of Lactobacillus plantarum Formulation 3, (Lp-F3).

A shake flask was grown (30° C./200 rpm) for approximately 8 hrs to OD₆₀₀ of 1.5 which was then used to inoculate a production vessel (100 L). Lactobacillus plantarum was fermented at 30° C., pH 6.5 for 16 hrs on Soytone-MRS+5% glucose to OD₆₀₀ₙₘ 20 followed by heat-inactivation of the cells at 70° C. for 20 min. The inactivated culture was cooled down to 30° C. after which it was ready to be harvested. The harvested heat-inactivated Lactobacillus plantarum cells were centrifuged yielding approximately 30 g per pellet per liter of culture. The pellet was washed in 1×PBS with approximately ⅕ of the initial volume and centrifuged again. The cells were resuspended in 49 mM KH₂PO₄, 11 mM Na₂PO₄, 155.2 mM NaCl up to a final concentration of 1×10¹¹ cells/mL and frozen at −20° C. Isolation of a whole cell product was confirmed by cell count by hemocytometry before and after inactivation and inactivation was confirmed by 48 hour incubation in Soytone-MRS broth and agar plates.

Preparation of Lactobacillus plantarum Formulation 4, (Lp-F4).

The method of preparation of Lactobacillus plantarum formulation 3 was used, however after centrifugation the cells were re-suspended in 49 mM KH₂PO₄, 11 mM Na₂PO₄, 155.2 mM NaCl plus 10% Trehalose up to a final concentration of 1×10¹¹ cells/mL and frozen at −20° C.

### Preparation of Spray Dry Drug Product

The frozen bulk drug substance of concentrated cells at a concentration of 1·10¹¹ cells/mL and in 49 mM KH₂PO₄, 11 mM Na₂PO₄, 155.2 mM NaCl, 10% Trehalose, were thawed at ambient temperature and spray-dried on a PSD-1 scale spray dryer to an average D(v.05) particle size ranges of 20 to 30 m. Spray-dried material 10-50 mgs is packaged under low % humidity in Aptar® intranasal dry power delivery device, with desiccants and overwrapped with foil-pouch.

### Toll-Like/NOD-Like/C-Type Lectin Receptor and THP1-Dual Ligand Screening.

Toll-Like Receptor (TLR), NOD-Like Receptor (NLR) and C-Type Lectin Receptor (CLR) stimulation were tested by assessing NF-κB activation in HEK293 cells expressing a given TLR, NLR or CLR. The activity of the test articles were tested on seven different human TLRs (TLR2, 3, 4, 5, 7, 8 and 9), two different human NLRs (NOD1 and NOD2) and two human CLRs (Dectin-1a and Dectin-1b) as potential agonists. The test articles were additionally evaluated in THP1-Dual cells, a human monocytic cell line that naturally expresses many pattern-recognition receptors (PRR). PRR stimulation in THP1-Dual cells was tested by assessing NF-κB or IRF activation. The test articles were evaluated at one concentration and compared to control ligands (see list below). This step was performed in triplicate.

TLR/NLR/CLR: Control Ligands
hTLR2: HKLM (heat-killed Listeria monocytogenes) at 108 cells/mL
hTLR3: Poly(I:C) HMW at 1 μg/mL
hTLR4: E. coli K12 LPS at 100 ng/mL
hTLR5: S. typhimurium flagellin at 1 μg/mL
hTLR7: CL097 at 1 μg/mL
hTLR8: CL075 at 1 μg/mL
hTLR9: CpG ODN 2006 at 1 μg/mL
hNOD1: C12-iE-DAP at 1 μg/mL
hNOD2: L18-MDP at 100 ng/mL
hDectin-1a and hDectin-1b:
   WGP Soluble (β-glucan from S. cerevisiae) at 10 ng/mL
   Curdlan at 100 μg/mL
   Zymosan Depleted (hot alkali treated S. cerevisiae) at 5 μg/mL

THP1-Dual: Target Ligand Concentration
RIG-I: Poly(dG:dC)/LyoVec™ at 5 μg/mL
RIG-I: 5′ppp-dsRNA/LyoVec™ at 10 μg/mL
Type I IFN: hIFNα at 103 IU/mL
TLR2: HKLM at 108 cells/mL
TLR3: Poly(I:C) at 1 μg/mL
TLR4: E. coli K12 LPS Ultra Pure at 1 μg/mL
TLR5: S. typhimurium flagellin Ultra Pure at 1 μg/mL
TLR7/8: R848 at 10 μg/mL
TLR9: CpG ODN 2006 at 1 μg/mL
NOD1: C12-iE-DAP at 10 μg/mL
NOD2: L18-MDP at 10 μg/mL
NF-κB: TNFα at 1 μg/mL

NF-κB Negative Controls
HEK293/Null1: TNFα at 100 ng/mL
   Control for human TLR2, 3, 5, 8, 9 and NOD1
HEK293/Null1-k: TNFα at 100 ng/mL
   Control for human TLR7
HEK293/Null1-v: TNFα at 100 ng/mL
   Control for human Dectin-1a and Dectin-1b
HEK293/Null2: TNFα at 100 ng/mL
   Control for human TLR4 and NOD2

Test Articles

Test Articles

| Article 1: | Lactobacillus plantarum Fermentor Heat-inactived |
|---|---|
| Stock Concentration: | 10¹¹ CFU/mL |
| Volume: | 1.5 mL × 2 |
| Storage Condition: | −80° C. |
| Final Concentration: | 10⁸ CFU/mL |
| Article 2: | Lactobacillus plantarum PBS Heat-inactivated |
| Stock Concentration: | 10¹¹ CFU/mL |
| Volume: | 1.5 mL × 2 |
| Storage Condition: | −80° C. |
| Final Concentration: | 10⁸ CFU/mL |
| Article 3: | PBS + 20% Glycerol |
| Stock Concentration: | N/A |
| Volume: | 10 mL |
| Storage Condition: | −80° C. |
| Final Concentration: | 1/10 |
| Article 4: | Heat Killed E. coli 0111:B4 |
| Stock Concentration: | 10¹⁰ CFU/mL |
| Volume: | 1 mL |
| Storage Condition: | −20° C. |
| Final Concentration: | 10⁸ cells/mL |
| Article 5: | Heat killed Lactobacillus rhamnosus |
| Stock Concentration: | 10¹⁰ CFU/mL |
| Volume: | 1 mL |
| Storage Condition: | −20° C. |
| Final Concentration: | 10⁸ cells/mL |

Preparation of Test Articles
Article 1: Lactobacillus plantarum formulation 1, (LP-F1) at 10E11 cells/mL
   Prepare 10E10 cells/mL by adding 0.05 mL of Article 1 at 10E11 cells/mL to 0.450 mL of Article 3 (PBS+20% glycerol) and vortex.
   Prepare 10E9 cells/mL by adding 0.15 mL of Article 1 at 10E10 cells/mL to 1.350 mL of Article 3 (PBS+20% glycerol) and vortex.
Article 2: Lactobacillus plantarum formulation 2, (LP-F2) at 10E11 cells/mL
   Prepare 10E10 cells/mL by adding 0.05 mL of Article 2 at 10E11 cells/mL to 0.450 mL of Article 3 (PBS+20% glycerol) and vortex.
   Prepare 10E9 cells/mL by adding 0.15 mL of Article 2 at 10E10 cells/mL to 1.350 mL of Article 3 (PBS+20% glycerol) and vortex.
Article 3: PBS+20% Glycerol is tested at 1/10.
Article 4: Heat Killed E. coli 0111:B4
   Prepare 10E9 cells/mL by adding 0.15 mL of Article 4 at 10E10 cells/mL to 1.350 mL of sterile endotoxin-free water and vortex.
Article 5: Heat killed Lactobacillus rhamnossus
   Prepare 10E9 Prepare 10E9 cells/mL by adding 0.15 mL of Article 5 at 10E10 cells/mL to 1.350 mL of sterile endotoxin-free water and vortex.

### TLR/NLR/CLR.

The Secreted Embryonic Alkaline Phosphatase (SEAP) reporter was under the control of a promoter inducible by the transcription factor NF-κB. This reporter gene allows the monitoring of signaling through the TLR, NLR or CLR based on the activation of NF-κB. In a 96-well plate (200 μL total volume) containing the appropriate cells (50,000-75,000 cells/well), 20 μL of the test article or the positive control ligand was added to the wells. The media added to the wells was designed for the detection of NF-κB induced SEAP expression. After a period of 16-24 hr incubation the Optical Density (OD) was read at 650 nm on a Molecular Devices SpectraMax 340PC absorbance detector.

### THP1-Dual.

THP1-Dual cells were derived from THP-1, a human monocyte cell line that naturally expresses many pattern-recognition receptors. THP1-Dual cells have been stably integrated with two inducible reporter constructs that allow the simultaneous study of the NF-κB and IRF pathways.

### NF-κB Pathway.

The Secreted Embryonic Alkaline Phosphatase (SEAP) reporter was under the control of a promoter inducible by the transcription factor NF-κB. This reporter gene allows the monitoring of signaling through the TLR or NLR, based on the activation of NF-κB. In a 96-well plate (200 μL total volume) containing the appropriate cells (100,000 cells/well), 20 μL of the test article or the positive control ligand was added to the wells. After a 16-24 hr incubation, SEAP production was assayed from the supernatant of the induced cells. The Optical Density (OD) was read at 650 nm on a Molecular Devices SpectraMax 340PC absorbance detector after an additional 3 hour incubation period.

### IRF Pathway.

The secreted luciferase reporter was under the control of a promoter inducible by IRF transcription factors. This reporter gene allows the monitoring of signaling through type 1 IFNs, RIG-I-Like Receptors and Cytosolic DNA Sensors. In a 96-well plate (200 μL total volume) containing the appropriate cells (100,000 cells/well), 20 μL of the test article or the positive control ligand was added to the wells. After 16-24 hr incubation, activation of the IRF pathways were assayed using a proprietary luciferase detection assay. Luciferase activity was assayed from the supernatant of the induced cells, and the Relative Luminescence Units (RLUs) were detected by a Promega GloMax Luminometer. The luciferase assay was performed in triplicate for each of the three screenings.

### DNA Microarray Target Preparation and Analysis.

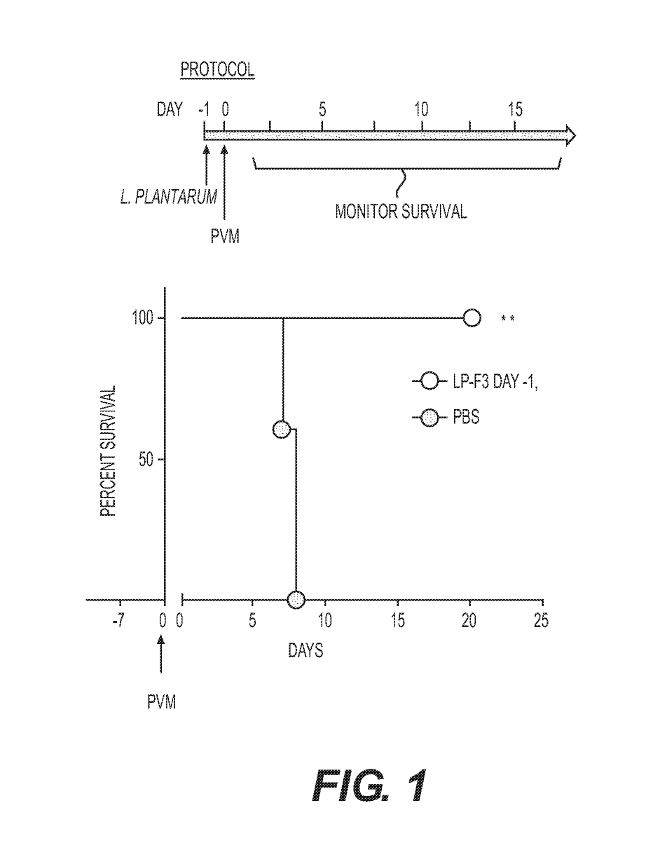

Eight-week old female BALB/c mice (all born on same day and shipped at same time from provider) were inoculated under isoflurane anaesthesia with live L. plantarum (50 μL of 2×10¹⁰ cfu/mL in pbs/bsa) or diluent control on day −14 and again on day −7 and then inoculated with 0.2 TCID50 units in 50 μL of PVM strain J3666 on day +14 or vehicle control (FIG. 1A). Each step of the study, including all mouse inoculations, RNA harvests to DNA microarray target preparation was designed and performed in a manner so as to avoid batch processing effects in the data due to mouse and sample type. Mouse inoculations, tissue harvest, RNA extraction, DNA target preparation batches were balanced between treatment and time. Lung tissues were harvested on days +17, +18, +19 and +20 and were snap frozen in liquid nitrogen. Samples (total 24, 6 mice per group) from mice that received two inoculations of L. plantarum or two inoculations of pbs/bsa diluent prior to virus or vehicle only challenge and harvested on day +19 were processed further for DNA microarray analysis. RNA extraction and target preparation were performed as described by Mackey-Lawrence and colleagues [2013 Infect. Immun. 81: 1460-1470] for all samples except RNA was extracted using RNeasy 96 well kit (Qiagen, Valencia, Calif.). Hybridization, fluidics and scanning were performed according to standard Affymetrix protocols (http://www.affymetrix.com) with the whole mouse genome 430 2.0 chip within the Genomics Unit of the Research Technologies Section (NIAID). Command Console (CC v3.1, http://www.Affymetrix.com) software was used to convert the image files to cell intensity data (cel files). All cel files, representing individual samples, were normalized by using the trimmed mean scaling method within expression console (EC v1.2, http://www.Affymetrix.com) to produce the analyzed cel files (chp files) along with the report files. The cel files were input into Partek Genomics Suite software (Partek, Inc. St. Louis, Mo., v6.6-6.12.0907) and quantile-normalized to produce the principal components analysis (PCA) graph and dendrogram. An ANOVA was performed within Partek to obtain multiple test corrected p-values using the false discovery rate method (FDR) as described by Klipper-Aurbach and colleagues [1995 Med. Hypotheses 45: 486-490] at the 0.05 significance level which were combined with fold change values for each comparison of interest.

The full DNA microarray data set for the biomarkers of Lactobacillus-mediated protection, a subset of which was presented in the Examples and Figures here in, have been deposited in NCBI's Gene Expression Omnibus and will be accessible through GEO series accession number GSE66721.

### Virus Titer Determination.

cDNA was generated from total RNA from mouse lung tissue via a dual standard curve qRT-PCR method targeting the PVM SH gene and mouse GAPDH; this assay generates absolute copy numbers per copy GAPDH (PVMSH/GAPDH) as previously described by Percopo and colleges [2014 Methods In Mol. Bio., Chapter 23, Walsh, G. A., ed. Humana Press].

### Cytokine Analysis.

Cytokines were detected from cDNAs generated from total lung RNA from mouse lung tissue as previously described [26]. Detection of transcripts encoding CCL2, CXCL10 and IL-6 was carried out using concentrated primer-probe sets Mm00441242_M1, Mm00445235_m1, and Mm00446191_m1, respectively (Advanced Biotechnologies, Inc.). Relative quantification (RQ) was determined via normalization to expression of mouse GAPDH (GADPH-vic primer-probe 4308313); one experimental replicate of the n=6 from the group that received L. plantarum at day −14 and at day −7, followed by PVM at day +17 samples (FIG. 1A) was normalized to 1.0. Cytokine ELISAs (R&D Systems) were performed on clarified homogenates of lung tissue and corrected for total protein by BCA assay (Pierce) as previously described by Garcia-Crespo and colleagues [2013 Antiviral Res. 97: 270-279.

### Histology.

Tissue sections prepared from 10% phosphate-buffered formalin-fixed lung tissue were stained with hematoxylin and eosin (H&E; Histoserv, Germantown, Md.)

### Example 1

A Single Intranasal Inoculation with L. plantarum One Day Prior to PVM Challenge Results in Survival in Response to an Otherwise Lethal Infection.

Eight week old BALB/c mice were inoculated intranasally with 50 μL of 2×10¹⁰ cells/mL of L. plantarum, formulation 3, (Lp-F3) or PBS with 0.1% BSA alone on day −1 and received a 50 μL intranasal inoculation with PVM (0.2 TCID₅₀ units/mL) on day 0. The mice were monitored for survival out to day 21 (FIG. 1). A single intranasal inoculation with L. plantarum one day prior to PVM challenge results in full protection against the lethal sequelae of PVM. From this result we conclude that there is a rapid induction of protective responses following intranasal inoculation with L. plantarum (**p<0.01 log rank).

### Example 2

A Single Intranasal Inoculation with L. plantarum One Day after PVM Challenge Results in Survival from an Otherwise Lethal Infection.

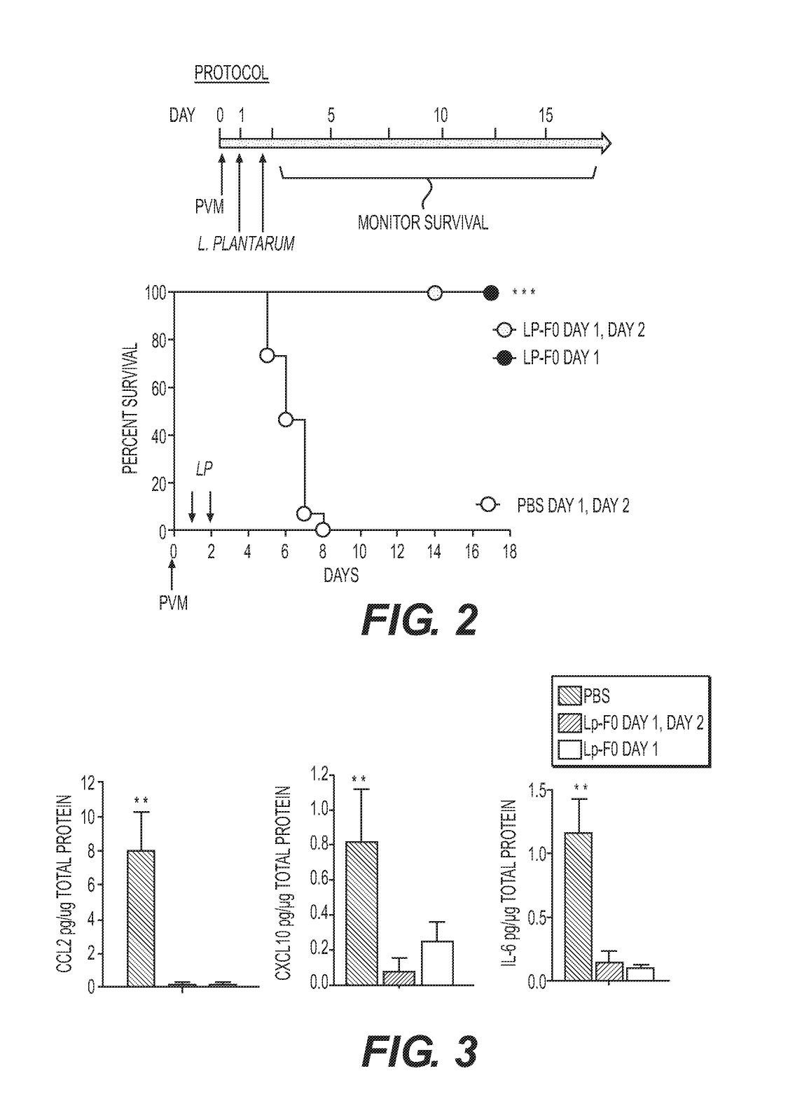

Eight week old BALB/c mice were intranasally inoculated with 50 μL PVM on day 0 and received a 50 μL intranasal inoculation with 2×10⁹ cells/mL L. plantarum, LP-F0 or PBS/BSA on day +1 or on days +1 and +2. The mice were monitored for survival out to day 18 (FIG. 2). In contrast to the 100% mortality observed in the group inoculated on day +1 and day +2 with PBS/BSA, a single intranasal inoculation on day +1 only or one inoculation each on days +1 and +2 with L. plantarum after PVM challenge resulted in full protection against the lethal sequelae of this infection (***p<0.001 log rank).

### Example 3

Intranasal Inoculation with L. plantarum after Virus Challenge Reduces Virus Recovery and Suppresses Inflammation.

Eight week old BALB/c mice were intranasally inoculated with 50 μL PVM on day 0 followed by 50 μL intranasal inoculations with 2×10⁹ cells/mL L. plantarum, Lp-F0 or PBS/BSA on day +1 or on days +1 and +2 (as in FIG. 2). Compared to the control cohort (mice that received PBS/BSA), cytokine biomarkers CXCL2, CCL2, and IL-6 were significantly suppressed in the mice that were inoculated with L. plantarum on day +1 and on days +1 and +2 after inoculation with PVM on day 0, FIG. 3 (**p<0.01, Mann-Whitney U-test).

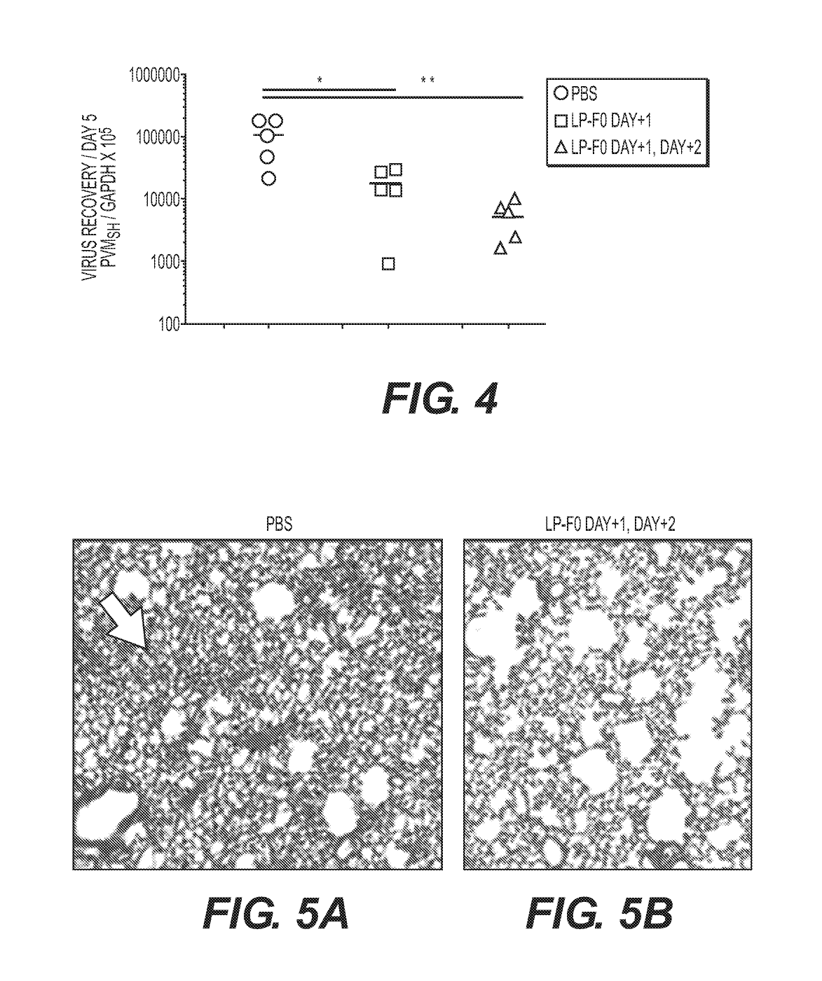

PVM virus recovery and cytokines were measured on day +5 after virus challenge. Viral load, although not a direct determinant of survival [Gabryszewski et al., 2011 J. Immunol. 186: 1151-1161] was also diminished among mice that received L. plantarum on day +1 and on days +1 and +2 after challenge with PVM, FIG. 4 (*p<0.05, **p<0.01, Mann-Whitney U-test).

Lung tissue from mice that received diluent control only rather than L. plantarum on days +1 and +2 after PVM challenge displayed prominent alveolitis and congestion, indicating initial onset of edema (FIG. 5a—PBS treated; FIG. 5b—L. plantarum treated).

In summary, post-virus challenge administration of L. plantarum had similar physiologic effects with respect to the suppression of the cytokine response and the decrease in viral load as was observed in response to L. plantarum priming prior to PVM [Gabryszewski et al., 2011 J. Immunol. 186: 1151-1161]. This finding serves to expand the scope of this discovery, and to enlarge the utility and applicability of a potential product.

### Example 4

Lactobacillus-Mediated Suppression of Virus-Induced Chemokines CCL2, CXCL10, and IL-6 is Directly Associated with Survival.

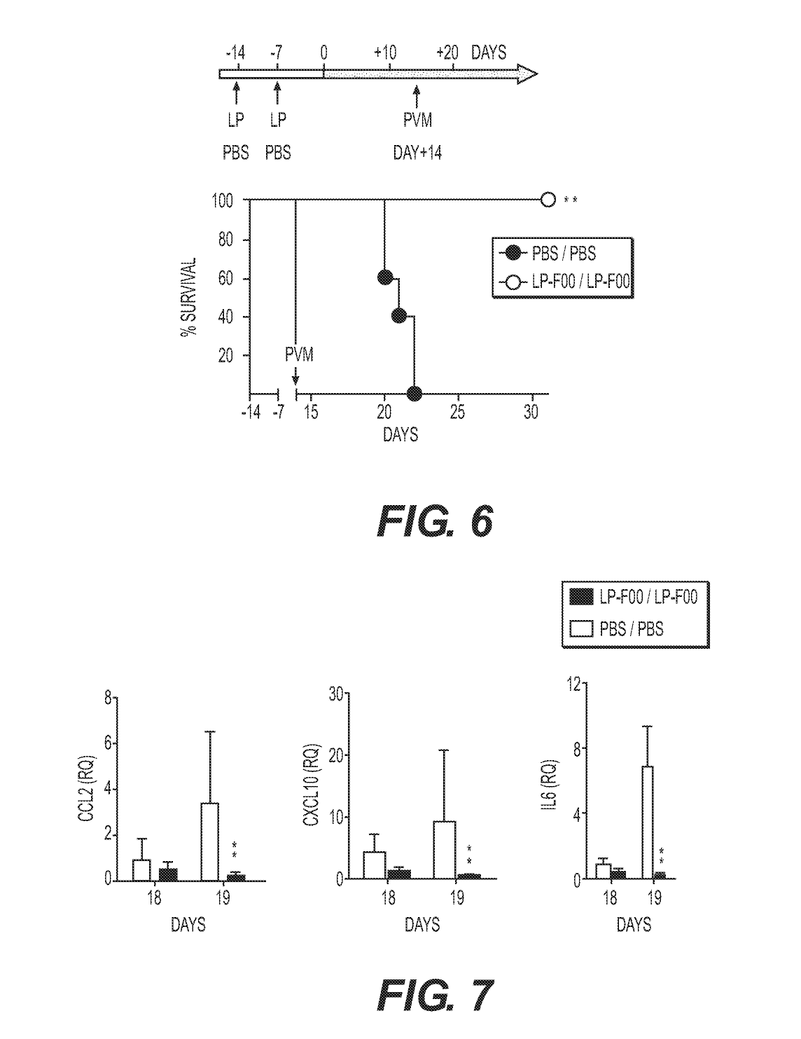

Mice were primed on days −14 and −7 with 10⁹ cells L. plantarum, Lp-F00 or control (PBS/BSA) and challenged with a lethal inoculum of PVM on day +14. As anticipated, the PVM infection was fully lethal among mice in the control group, whereas 100% of the L. plantarum-primed mice survived, FIG. 6 (**p<0.01 log rank). Survival in the Lactobacillus-primed group was associated with profound suppression of proinflammatory cytokines CCL2, CXCL10, and IL-6 induced in response to virus infection in the control (PBS/BSA) primed mice, FIG. 7 (**p<0.01, Mann-Whitney U-test).

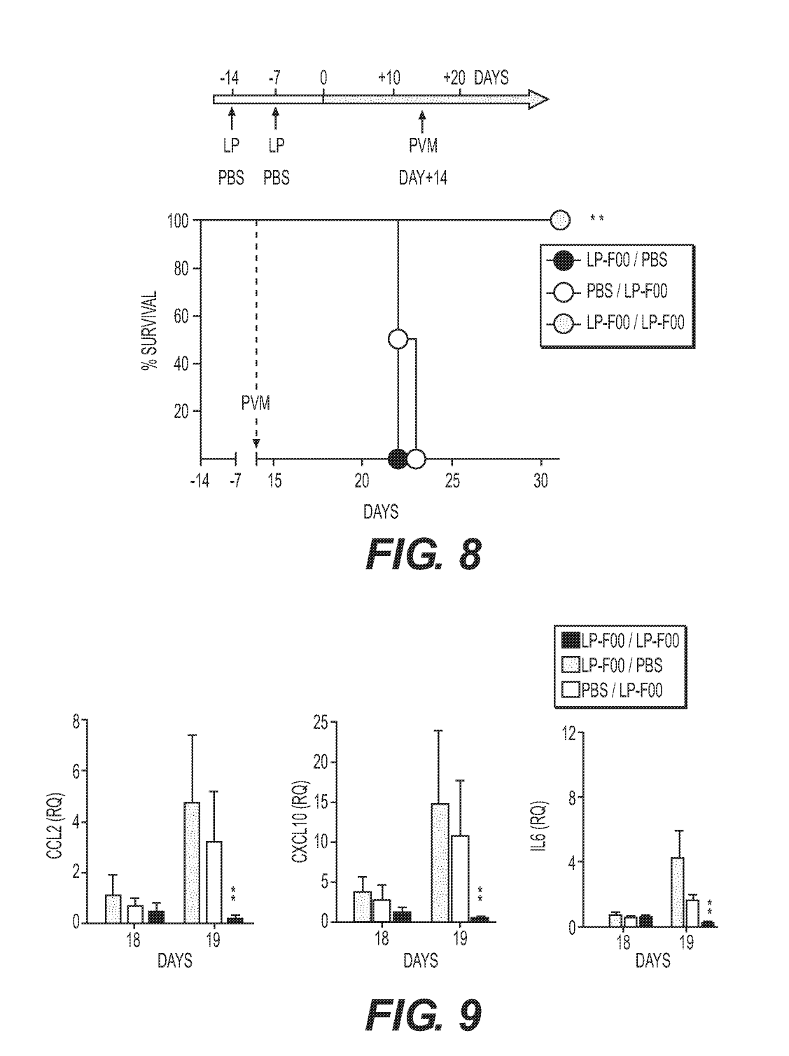

In order to assess further the relationship between survival and cytokine suppression associated with L. plantarum-priming, mice were primed on day −14 or on day −7 alone, or on both days −7 and −14 with 10⁹ cells L. plantarum (Lp-F00) followed by challenge with a lethal inoculum of PVM on day +14. As shown, only those animals that were intranasally inoculated with L. plantarum on both days −14 and −7 were protected from the lethal sequelae of PVM infection, FIG. 8 (**p<0.01 log rank).

The suppression of proinflammatory cytokines CXCL10, CCL2, and IL-6 is observed only in response to the priming regimen that promotes survival, ie., intranasal inoculation with L. plantarum on both days −14 and −7, FIG. 9 (**p<0.01, Mann-Whitney U-test).

In summary, consistent with the microarray expression findings (Table 1), mice that received two intranasal inoculations with L. plantarum exhibit profound suppression of virus-induced CCL2, CXCL10, and IL-6 compared to mice primed with diluent alone. No significant suppression of any of these virus-induced cytokines was observed in response to single inoculations of L. plantarum, nor were mice protected from PVM infection. As such, we note the association of cytokine suppression with survival from an otherwise lethal PVM infection, and we identify suppression of virus-induced CCL2, CXCL10 and IL-6 as biomarkers for survival associated with L. plantarum administration to the respiratory mucosa.

### Example 5

L. plantarum Priming of the Respiratory Mucosa Protects Against the Lethal Sequelae of Infection with Influenza A/HK/68 (H3N2).

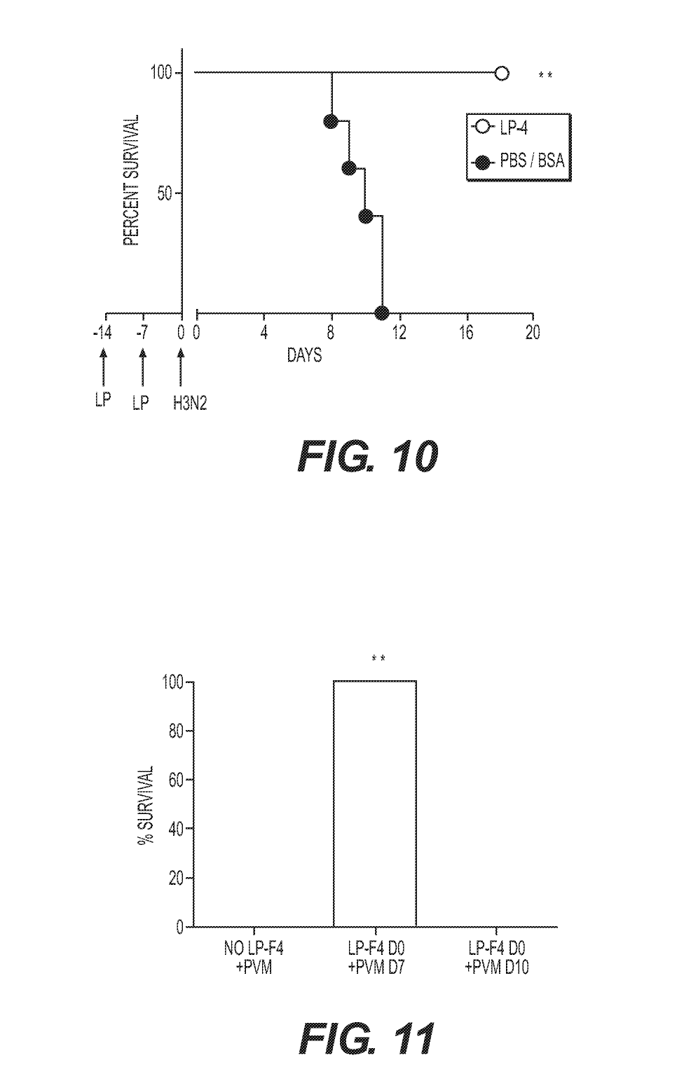

Protection elicited by priming with L. plantarum is not pathogen specific. BALB/c mice received 50 μL intranasal inoculations of 2×10¹⁰ cells/mL L. plantarum formulation 4, Lp-F4 or PBS/BSA on days −14 and day −7, followed by 50 μL intranasal inoculation of Influenza A (H3N2) on day 0. Survival was followed out to 21 days. In contrast to the 100% mortality that was observed in the control group, priming of the respiratory mucosa with L. plantarum resulted in full protection against an otherwise lethal inoculum of Influenza A (H3N2), FIG. 10 (**p<0.01 log-rank).

Thus, although the inflammatory response to respiratory virus infection is complex and can be refractory to standard therapy, intranasal inoculation with L. plantarum, when tested in two robust models of severe respiratory virus disease, is highly effective at suppressing a complex inflammatory response, and ultimately results in the protection against the lethal sequelae of respiratory virus infection.

### Example 6

A Single Intranasal Inoculation of L. plantarum Provides Limited Protection Against the Lethal Sequelae of PVM Infection.

Eight week old BALB/c mice were primed with L. plantarum formulation 4, (Lp-F4) 50 μL per inoculum, 2×10¹⁰ cells/mL on day 0, and challenged with PVM on days +7 and +10. Note that we have achieved full protection at +1 day post inoculation (FIG. 1). Here, we see that full protection is sustained through day +7 in response to a single inoculation. However, by day +10, protection elicited by a single inoculation with L. plantarum protection is lost, FIG. 11 (**p<0.01 log-rank).

### Example 7

Two Inoculations of L. plantarum Elicits Sustained Protection.

In this experiment we show that a two dose regimen of L. plantarum results in a dramatic increase in duration of protection over that provided by a single dose.

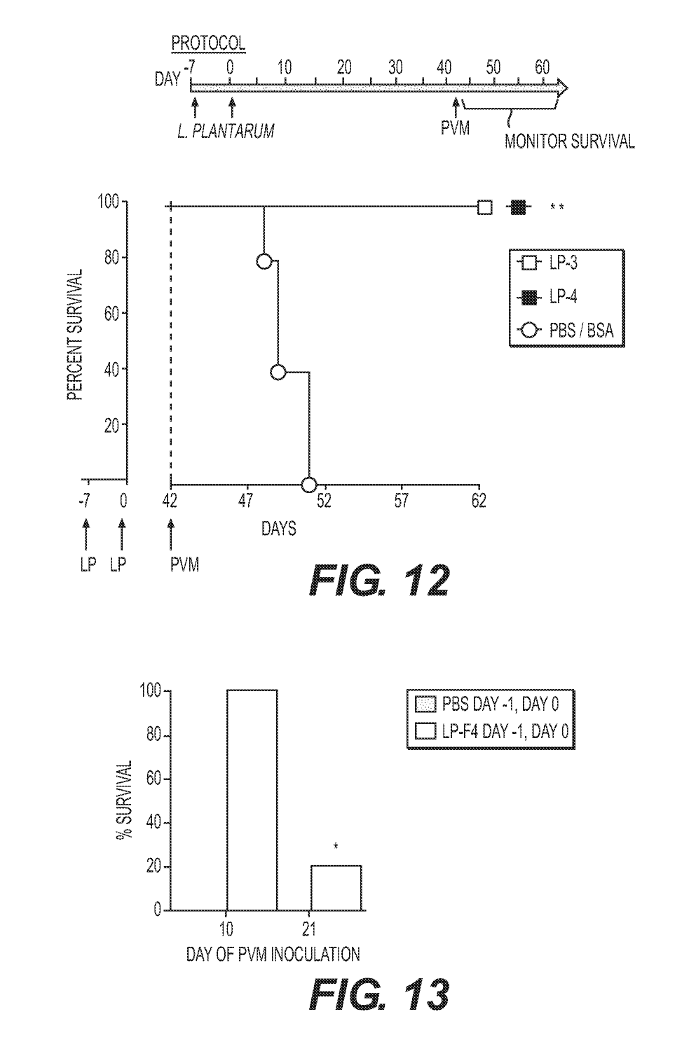

Eight week old BALB/c mice were inoculated with L. plantarum formulated either in PBS buffer (Lp-F3) or in PBS buffer containing 10% trehalose (Lp-F4) 50 μL per inoculum, 2×10¹⁰ cells/mL or PBS/BSA on days −7 and 0 and challenged with PVM on day 42. In contrast to the 100% mortality that was observed in the control group, priming of the respiratory mucosa with L. plantarum (Lp-F4) resulted in full protection against an otherwise lethal PVM infection, with inoculation delayed to day +42 after the final priming with L. plantarum on day 0, FIG. 12 (**p<0.01 log-rank).

### Example 8

In contrast to the sustained protection observed when two inocula are separated by one week (day −7 and day 0), protection elicited by L. plantarum priming on two consecutive days (days −1 and 0) was not significantly enhanced over that provided by a single inoculum.

Eight week old BALB/c mice were inoculated with L. plantarum formulation 4, (Lp-F4) 50 μL per inoculum, 2×10¹⁰ cells/mL or PBS/BSA on days −1 and 0 and challenged with PVM on days +10 or +21. Survival was monitored out to 21 days after each PVM inoculation. Despite receiving two inoculations of L. plantarum, full protection from lethal PVM infection was observed only up to 10 days, FIG. 13 (*p<0.05 log-rank), only slightly longer than that observed in response to a single inoculum (see FIG. 11).

In summary, examples 6, 7, and 8 demonstrate the importance of the interval between successive L. plantarum inoculations. With doses remaining constant per inoculation, protection provided in response to two inoculations on two consecutive days is only slightly longer than that observed in response to a single inoculation (see FIG. 11). Despite receiving two inoculations of L. plantarum, in this case, on two consecutive days (days −1 and 0), mice did not achieve the extended duration of protection that was observed when the two inoculations were administered one week apart (see FIGS. 12 and 13).

### Example 9

Sustained Protection can be Achieved with Repeat Once Monthly L. plantarum Inoculations.

Repeat inoculations were tested to determine if persistent full protection from lethal viral challenge could be sustained over many months.

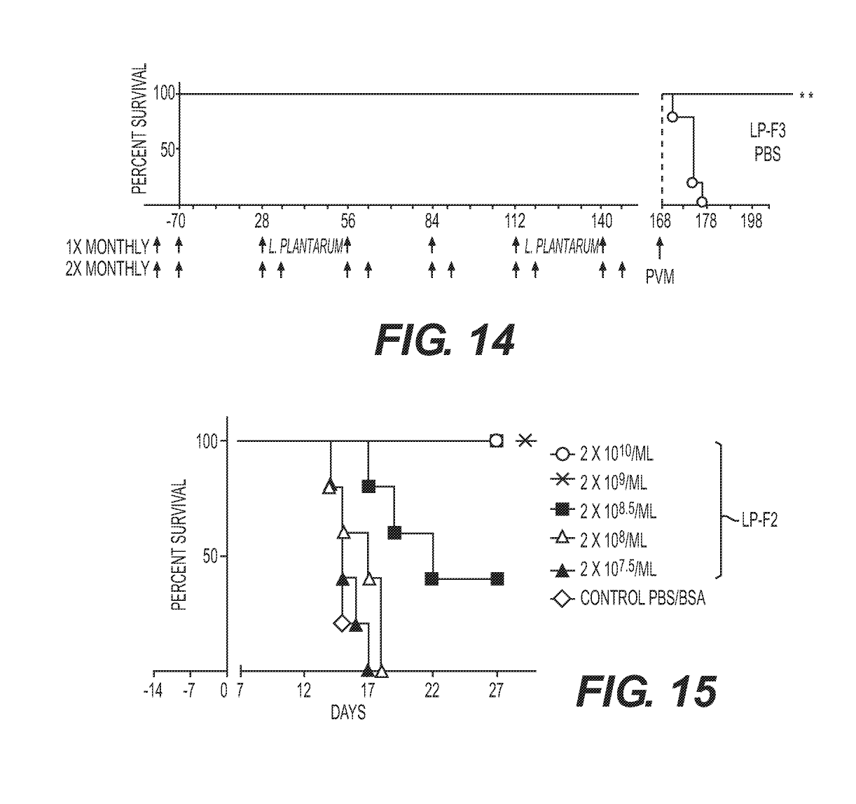

Eight week old BALB/c mice received a two dose loading protocol of L. plantarum formulation 3 (Lp-F3) 50 μL of 1.3×10⁹ cells/mL or PBS on days −7 and 0, which was followed by a maintenance protocol consisting of once monthly inoculations (once every 28 days) thereafter for 6 months. PVM challenge was suspended until 7 months (28 days following the last L. plantarum maintenance inoculation). Mice receiving once monthly maintenance inoculations sustained 100% survival compared 0% survival in the control group, FIG. 14 (**p<0.01 log-rank). Furthermore, an additional set of animals received a loading dose of L. plantarum on days −7 and 0 which was followed by a maintenance protocol consisting of twice monthly inoculations (once every 14 days) thereafter for 6 months. PVM challenge was suspended till 7 months (28 days following the last L. plantarum maintenance inoculation). Mice receiving once monthly maintenance inoculations sustained 100% survival compared 0% survival in the control group, FIG. 14 (**p<0.01 log-rank).

These findings demonstrate clearly that mice do not become inured to the impact of L. plantarum priming, nor is there any tachyphylaxis-type mechanism diminishing its long-term impact upon repeated exposure.

### Example 10

L. plantarum Promotes Dose-Dependent Survival Against PVM Infection.

Eight week old BALB/c mice (n=5 per group) were inoculated on days −14 and −7 with decreasing concentrations of inactivated L. plantarum formulation 2 (Lp-F2) at 50 μL per intranasal inoculum followed by PVM at day +7. L. plantarum concentrations ranged from 2×10¹⁰ to 2×10⁷0.5 cells/mL) diluted in PBS+0.1% BSA (PBS/BSA). The control mice receive PBS/BSA diluent on days −14 and −7 instead of L. plantarum. There is a clear dose relationship between the number of cells of L. plantarum in the inoculum and the effective degree protection against the lethal sequelae of PVM infection observed. The minimum dose required to sustain 100% survival under this L. plantarum priming/PVM challenge protocol is 50 μL of 2×10⁹ cells/mL which is equivalent to 1×10⁸ cells/mouse (FIG. 15).

### Example 11

L. plantarum is Effective Against a Strict Intranasal Influenza A/HK/68 H3N2 Infection.

Clinical symptoms (weight loss) can be measured in BALB/c mice provided with a strict intranasal inoculum (2.5 μL/nare) of Influenza A H3N2. This infection model was used to evaluate the impact of strict intranasal administration of L. plantarum. The inoculation volume used in this model limits the initial exposure of Lactobacillus plantarum and virus to the upper respiratory tract [Southam et al., 2002 Am J Physiol Lung Cell Mol Physiol. 282: L833-L839].

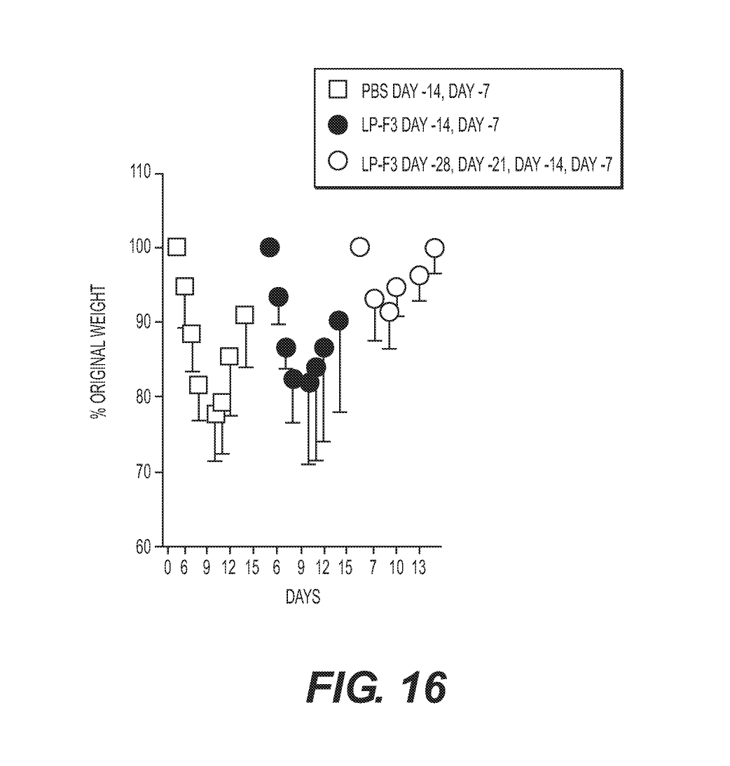

Eight week old BALB/c mice were inoculated with 5 μL L. plantarum formulation 3 (Lp-F3) 2.5 mL/nare at 10¹¹ cells/mL (dose equivalent to 5×10⁸ cells/mouse) either once weekly for two weeks (days −14 and −7) or once weekly for four weeks (days −28, −21, −14, and −7), followed by 5 μL (2.5 mL/nare) H3N2 on day 0. Weights of mice are as shown as % original weight. The control mice receive PBS/BSA diluent on days −14 and −7 instead of L. plantarum. Although mice primed with L. plantarum once weekly for two weeks show similar weight loss as the controls (nadir of 25-30% weight loss), the mice that were primed with L. plantarum once per week for 4 weeks showed a relatively minimal weight loss of ˜10% original body weight promoted by H3N2 infection (FIG. 16).

### Example 12

L. plantarum Activates Toll Like Receptor 2 (TLR2) and Nucleotide Binding Oligomerization Domain-Containing Protein 2 (NOD2) Signaling In Vitro.

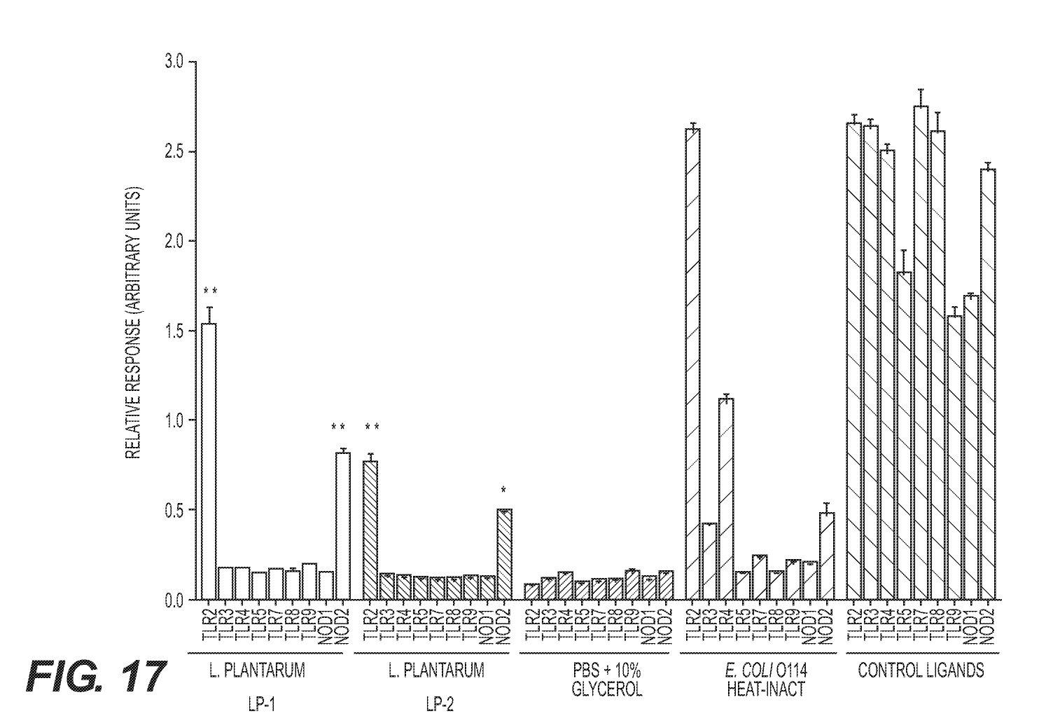

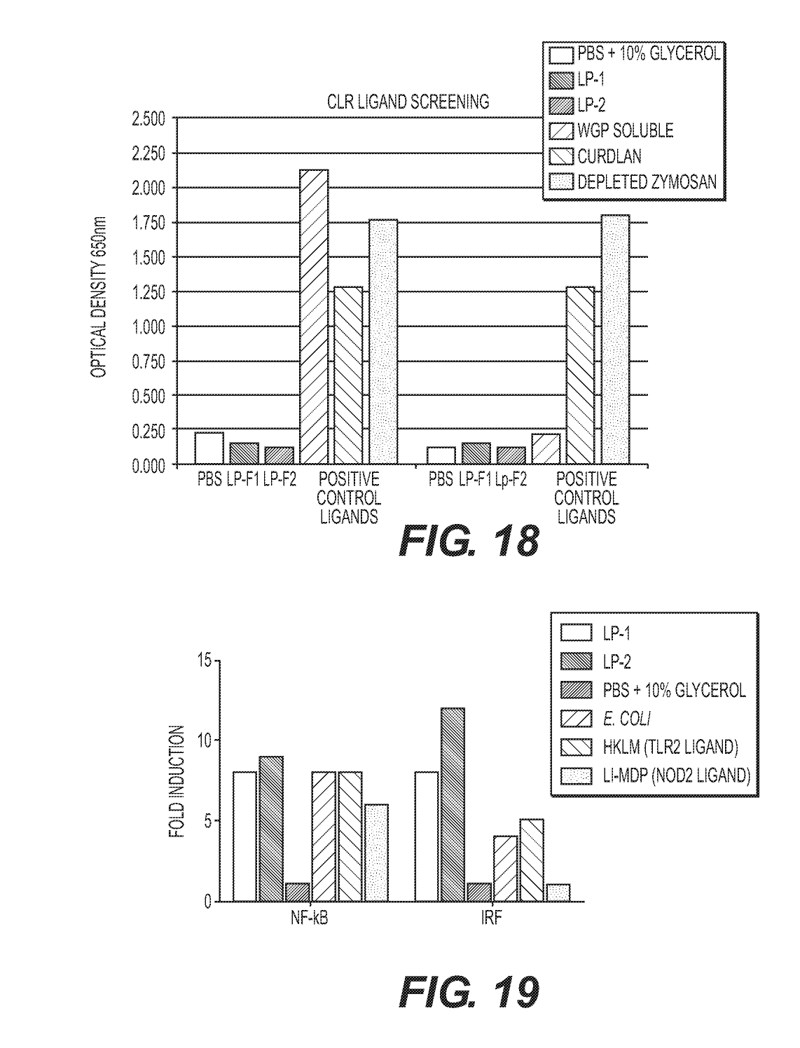

As part of an exploration of the mechanism of Lactobacillus-induced protection against the inflammatory sequelae of respiratory viral infection, we performed a screen to identify interactions between Lactobacillus plantarum (Lp-F1 and Lp-F2) and a panel of human toll like receptors (TLRs), nucleotide-binding oligomerization domain receptors (NLRs), and C-type lectin receptors (CLRs). Stably transfected HEK293 cell reporter lines express individual human recognition receptors (PRRs) and signal through TLRs, NLRs or CLRs based on activation via the transcriptional regulator, NF-κB. Relative response was determined via detection of secretory alkaline phosphatase (A650). Following co-incubation with these stably transfected HEK293 cell reporter lines, L. plantarum, at a final concentration of 1×10⁸ cells/mL, was shown to interact with and promote signaling primarily via pattern recognition receptors TLR2 and NOD2 at 20-fold and 6-fold over diluent control, respectively (FIG. 17). No signaling elicited by L. plantarum via CLR receptors was observed (FIG. 18). Other than TLR2 and NOD2 we observed no additional interactions, although PRR positive control ligands were uniformly reactive. FIGS. 17 and 18 shown are the combined results three experiments.

### Example 13

L. plantarum can Signal Via Both NF-κB and IRF Pathways in the THP Human Monocyte Cell Line.

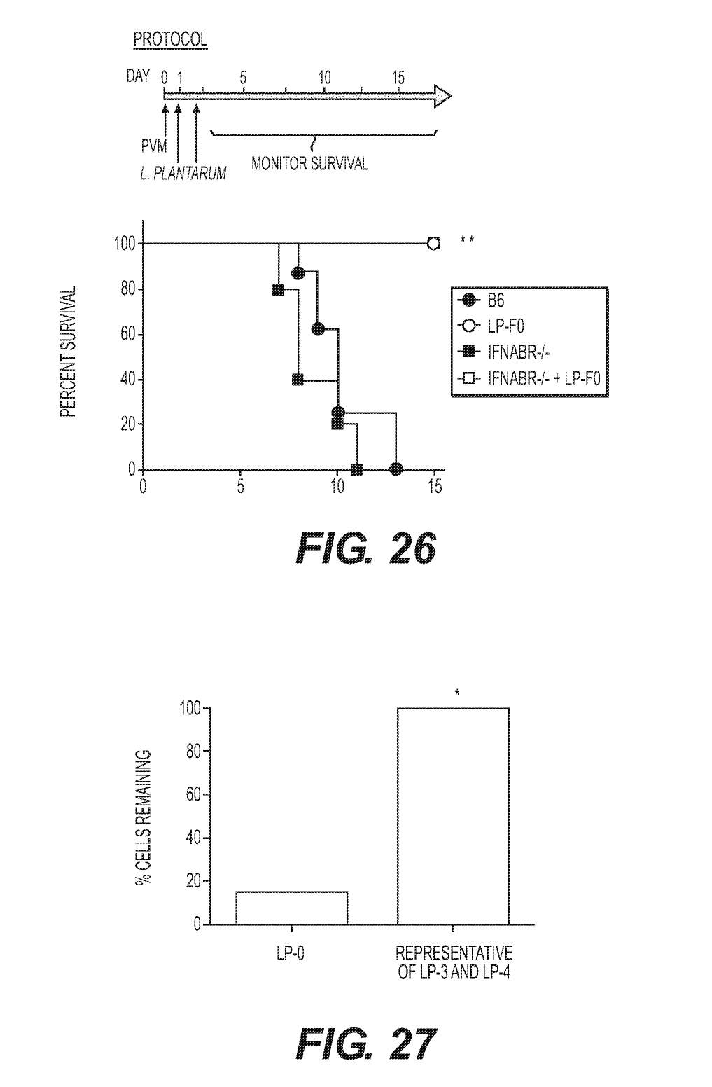

Signaling in response to L. plantarum (Lp-F1 and Lp-F2) at a final concentration of 1×10⁸ cells/mL was also evaluated in a THP1-Dual reporter cell line in which both NF-κB and IRF pathways were active. THP1 is a human monocyte cell line that naturally expresses multiple pattern-recognition receptors including hTLR2 and hNOD2. The N-κB reporter monitors of signaling through the TLRs and NLRs, based on the activation of NF-κB. The IRF pathway monitors signaling through type 1 IFNs, RIG-I-Like receptors and cytosolic DNA sensors. As shown, L. plantarum can activate both signaling pathways at 8-12 fold over baseline levels (FIG. 19). Although L. plantarum can activate IRF in vitro, additional studies carried out in mice devoid of the receptor for type I interferons (IFNαβR^{-/-} mice; Mueller et al., 1994 Science 264: 1918-1921) suggest that activation of this alone pathway is not sufficient to abrogate the protective effects of L. plantarum priming in vivo (see FIG. 26).

### Example 14

Mice Devoid of the Pattern Recognition Receptor, Toll-Like Receptor 2 (TLR2) or Nucleotide Binding Oligomerization Domain-Containing Protein 2 (NOD2) Remain Responsive to Lactobacillus plantarum “Prior to” or “after” PVM Challenge.

Although L. plantarum can activate the TLR2 and NOD2 receptors and activate their respective pathways, TLR2 or NOD2 single gene deletion is not sufficient to abrogate the protective impact of L. plantarum in vivo. Both TLR2 gene-deleted (TLR2^{-/-}) and NOD2 gene deleted (NOD2−/−) mice remain responsive to “priming” with Lactobacillus plantarum.

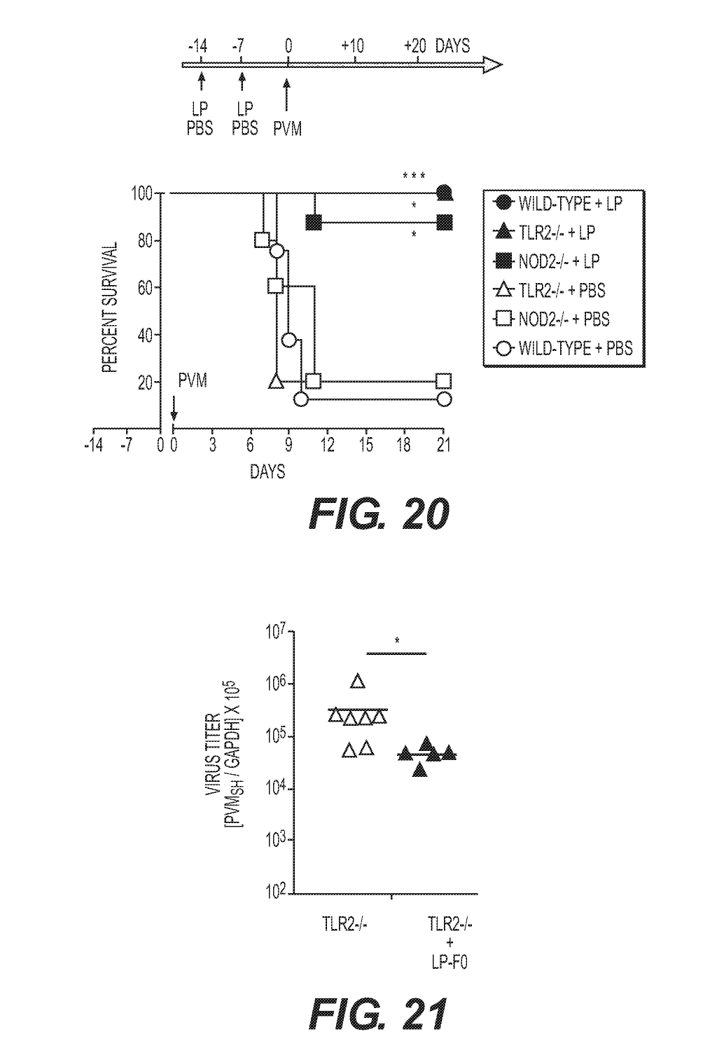

Six to 12 week old TLR2^{-/-} or NOD2^{-/-} single gene deleted mice and their wild type (C57BL/6) counterparts were inoculated on days −14 and −7 with inactivated L. plantarum Lp-F0 (50 microliters of 2×10⁹ cells/mL), followed by PVM at day 0. As with wild type, both TLR2^{-/-} and NOD2^{-/-} mice primed with L. plantarum were fully protected from the lethal sequelae of PVM infection in contrast to mice primed with diluent (pbs/bsa) only, FIG. 20 (***p<0.001; *p<0.05 log-rank).

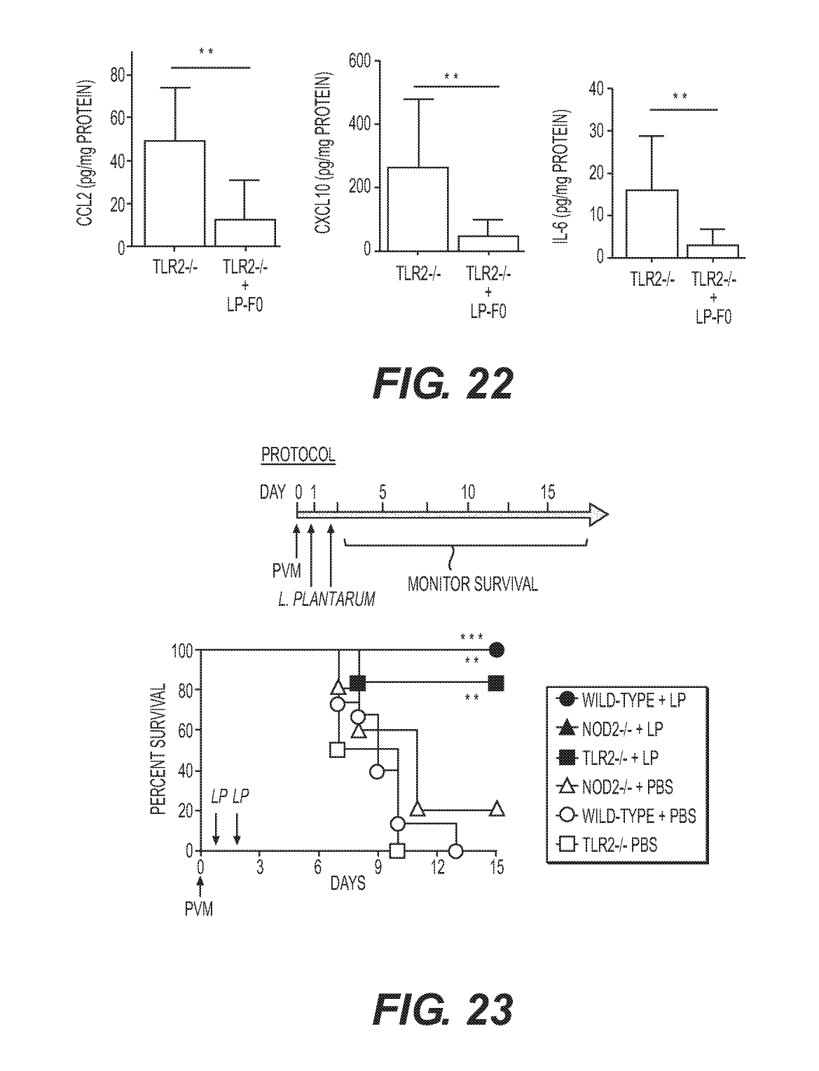

Priming of L. plantarum in TLR2^{-/-} mice resulted in diminished virus recovery (FIG. 21) as well as suppressed expression of virus-induced cytokines CCL2, CXCL10, and IL-6 in TLR2^{-/-} mice, FIG. 22 (*p<0.05; **p<0.01 Mann-Whitney U-test).

In summary single gene deleted TLR2^{-/-} and NOD2^{-/-} mice respond to priming in a manner that is indistinguishable from their wild type counterparts.

Analogous to the results observed in “priming” experiments, both TLR2 gene-deleted (TLR2^{-/-}) and NOD2 gene deleted (NOD2−/−) mice remain responsive to Lactobacillus plantarum “after” virus challenge and are protected against the lethal sequelae of PVM infection.

Six to 12 week old TLR2^{-/-,} NOD2^{-/-}, and their wild type counterpart, C57BL/6 mice, were inoculated with PVM on day 0 and treated with L. plantarum Lp-F0 (50 L of 2×10¹⁰ cells/mL) on days +1 and +2. As with wild type, both TLR2^{-/-} and NOD2^{-/-} mice who received L. plantarum after PVM challenge were protected from the lethal sequelae of PVM infection unlike mice primed with diluent (pbs/bsa) only, FIG. 23 (*p<0.05 log-rank; **p<0.01 log-rank).

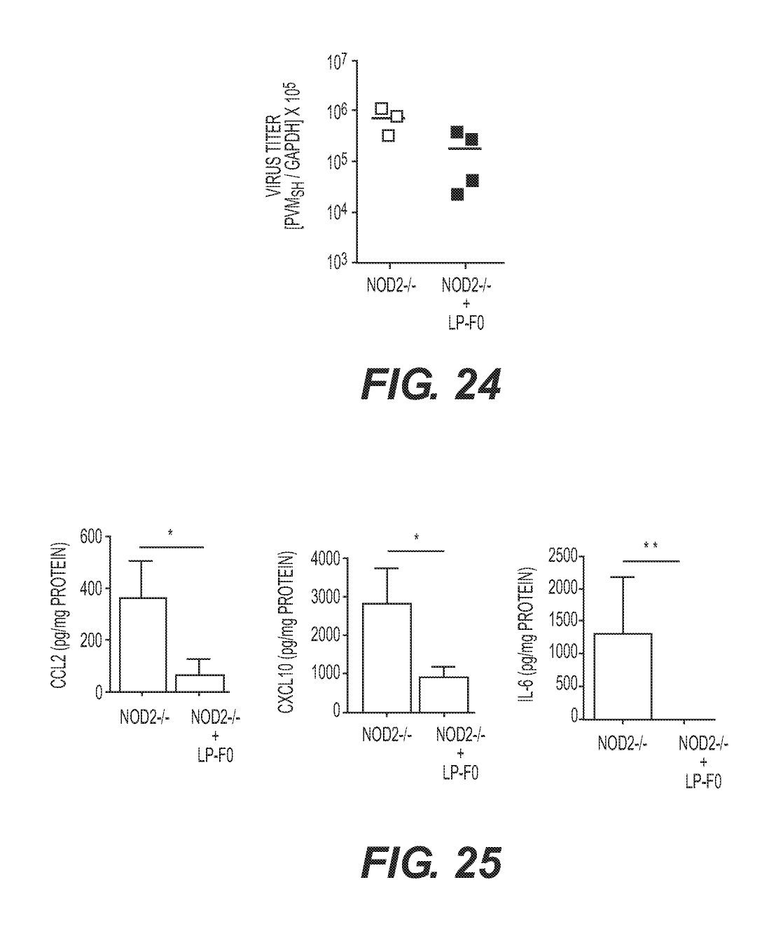

Analogous to what we have observed in wild-type mice, treatment of NOD2^{-/-} mice with L. plantarum on days +1 and day +2 resulted in diminished virus recovery (FIG. 24) as well as suppressed expression of virus-induced cytokines CCL2, CXCL10, and IL-6, FIG. 25 (*p<0.05; **p<0.01 Mann-Whitney U-test).

### Example 15

C57BL/6 Mice Devoid of the Receptor for Type I IFN Signaling Remain Responsive to Lactobacillus plantarum.

Although L. plantarum activates type I IFN pathways (see FIG. 19), deletion of the unique receptor for type I IFNs, IFNαβR, does not abrogate the protective effect of L. plantarum. As shown here, mice remain responsive to Lactobacillus plantarum and survive lethal PVM challenge.

Six to 12 week old wild-type c) and IFNαβR-gene-deleted mice were inoculated with PVM on day 0, and on days +1 and +2 with heat-inactivated L. plantarum (Lp-F0), 2×10¹⁰ cells/mL, 50 μL per intranasal inoculum. Both wild type C57BL/6 and IFNαβR-gene-deleted were fully protected from the lethal sequelae of PVM infection, FIG. 26 (**p<0.01 log-rank).

### Example 16

### An Optimized Heat Inactivation Yields Predominantly Whole Cells.

The fermentation, inactivation and isolation protocol was optimized to yield whole cell heat inactive L. plantarum formulations 3 and 4 (Lp-F3 and Lp-F4). FIG. 27 depicts the percent of whole cells remaining following inactivation conditions of Gabryszewski et al. 2011 compared the heat-inactivation process utilized for formulations Lp-F3 and Lp-F4 (*p<0.05 log-rank).

### Example 17

Glycerol Reduces the Efficacy of L. plantarum-Induced Protection Against Lethal PVM Infection.

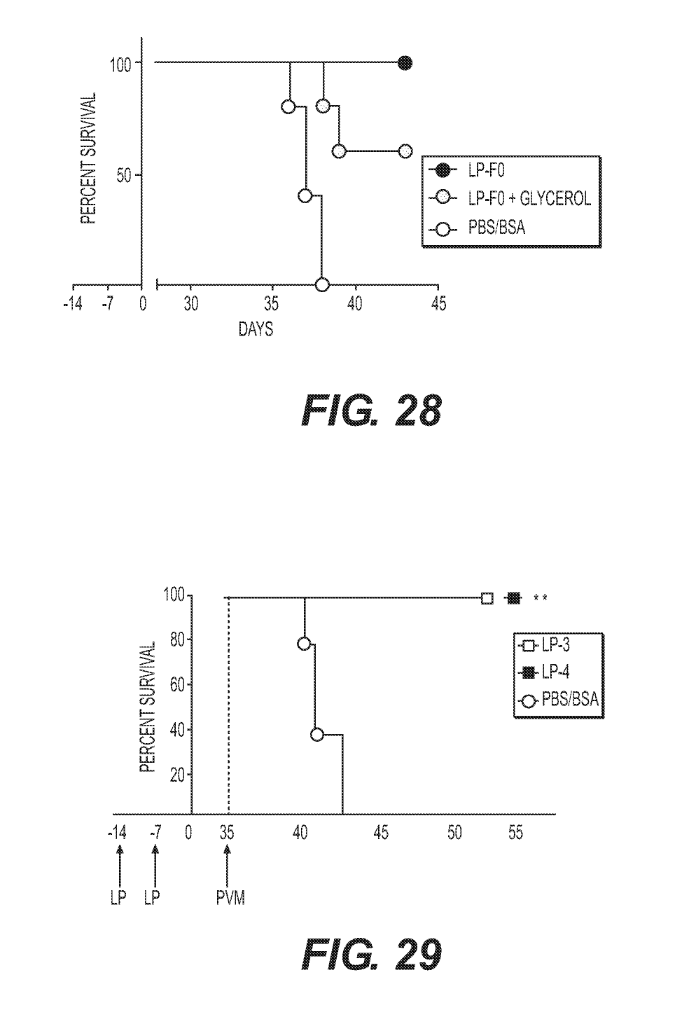

The L. plantarum stock was grown overnight in MRS medium in an Ehrlenmeyer flask, isolated, re-suspended in PBS and inactivated as described in Gabryszewski et al. 2011 (Lp-F0) and formulated at 10¹¹ cells/mL in PBS/0.1% BSA either with or without 20% glycerol. Mice were inoculated with L. plantarum (Lp-0) at days −14 and −7 (50 mL inoculum of 2×10¹⁰ cells/mL followed by PVM challenge at day 0. As shown, the addition of glycerol limits the efficacy of L. plantarum when administered at an otherwise fully protective dose when devoid of glycerol in the formulation (FIG. 28).

### Example 18

Whole Cell Heat-Inactivated L. plantarum Formulated in 10% Trehalose Retains Efficacy Against PVM Infection.

Eight week old BALB/c mice were inoculated with 50 μL L. plantarum formulated in 10% trehalose (Lp-F4) or L. plantarum formulated in PBS buffer (Lp-F3) on day −14 and again on day −7. Control mice received PBS only on day −14 and again on day −7. All animals received PVM on day +35. Trehalose (10%) buffer does not interfere with the efficacy of protection. Full survival (100%) was retained in the L. plantarum formulated in 10% trehalose and no differences were observed in efficacy between L. plantarum formulated in 10% trehalose compared to L. plantarum formulated in PBS was observed, FIG. 29 (**p<0.01 log-rank).

### Example 19

### Trehalose is an Effective Cryopreservative.

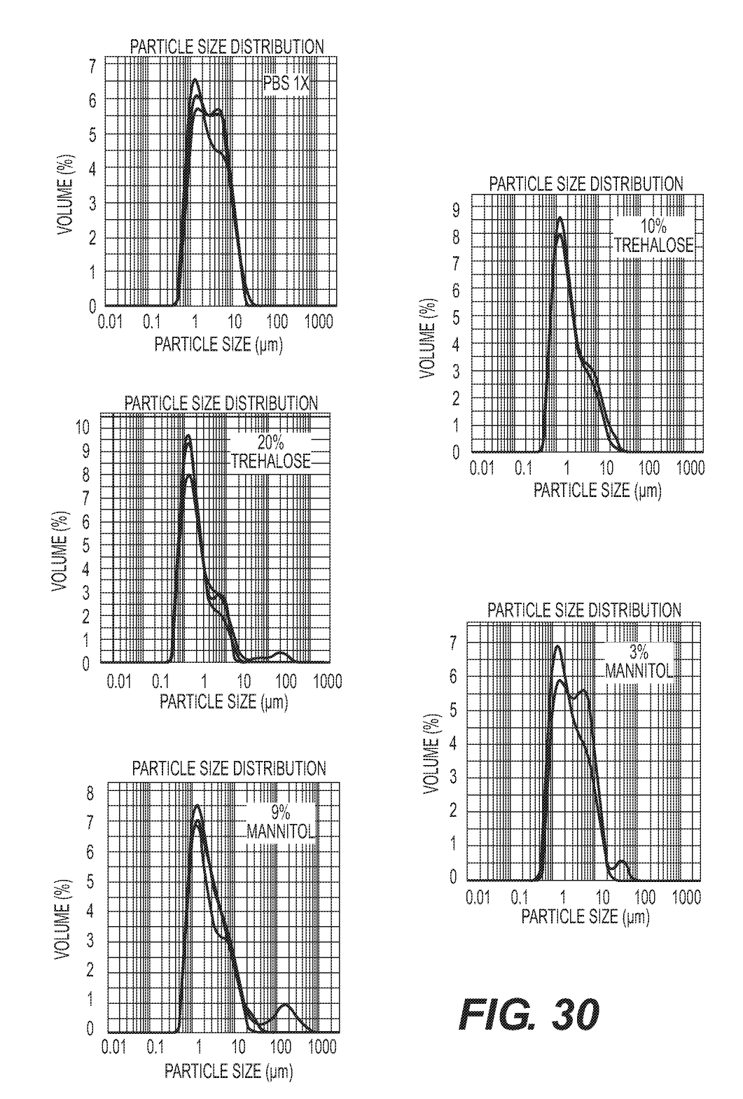

Heat-inactivated whole cell L. plantarum was formulated at 10¹¹ cells/mL in PBS with 10% or 20% trehalose, 3% or 9% mannitol or in PBS buffer alone. Each formulation was subject to three freeze (−20° C.) thaw (ambient temperature) cycles and measured for size distribution and cell lysis by static light scattering and picogreen assays respectively. As shown, 10% trehalose in PBS buffer prevented cell lysis as well as cellular aggregation and/or disaggregation after multiple freeze thaw cycles. Thus, a 10% trehalose/PBS buffer formulation is effective in maintaining the physical morphology of the fermented drug substance, specifically, heat-inactivated whole cell L. plantarum (Lp-F4) formulated at 10¹¹ cells/mL when frozen for purpose of storage and shipping (FIG. 30).

### Example 20

10% Trehalose is an Effective Bulking Agent for the Production of Spray Dried Heat-Inactivated L. plantarum.

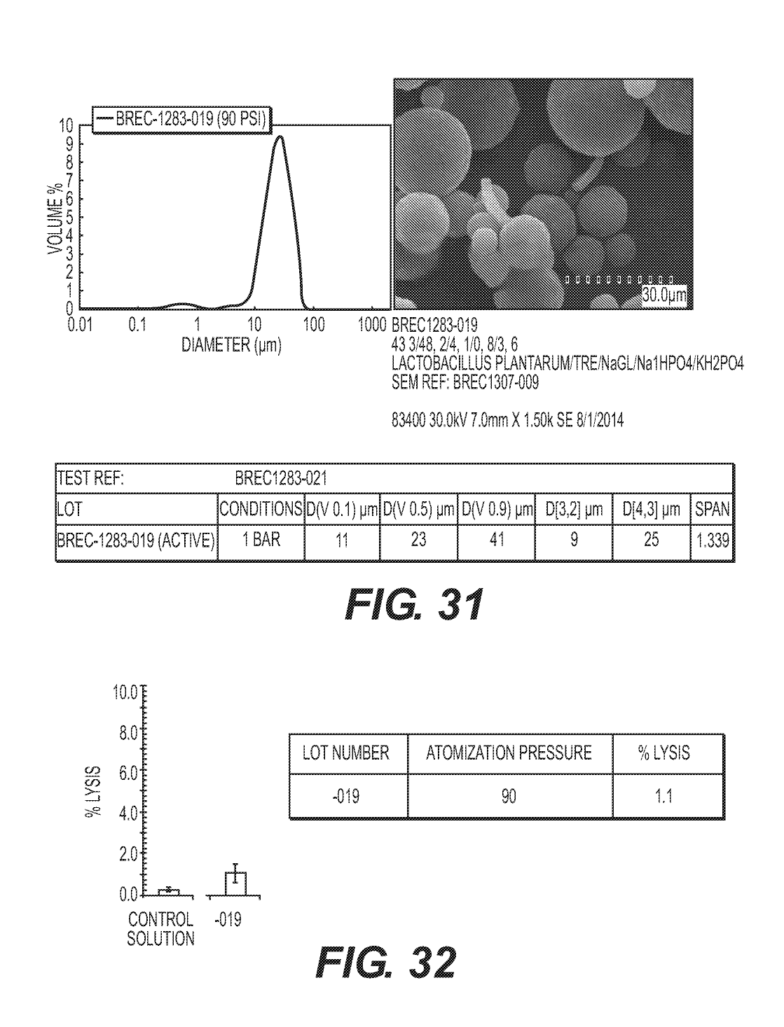

FIG. 31 depicts L. plantarum (Lp-F4) as a final spray dry powder drug product. The particle size of the spray dry power is depicted having an average particle size D (v 0.5) equal to 23 μm and a D (v 0.1) equal to 11 μm. The SEM image depicts spherical particles.

As shown in FIG. 32, the final product following the sequence of initial manufacturing, heat-inactivation, frozen shipping, thaw, and spray drying manufacturing does not result in disruption of the whole cell material. Liquid reconstitution of the dry powder drug product made from Lp-F4, followed by picogreen assay confirmed minimal lysis (1.1%) of L. plantarum cells in this representative example of the final drug product.

### Example 21

Protection Afforded by L. plantarum is not Mediated by IL-10 or IL-17A.

The administration of L. plantarum on days +1 and +2 after PVM challenge in both interleukin-10 gene-deleted (IL-10^{-/-}) and interleukin-17A (IL-17A^{-/-}) mice results in the full protection against lethal PVM challenge, from their wild-type (BALB/c and C57BL/6) counterparts, respectively.

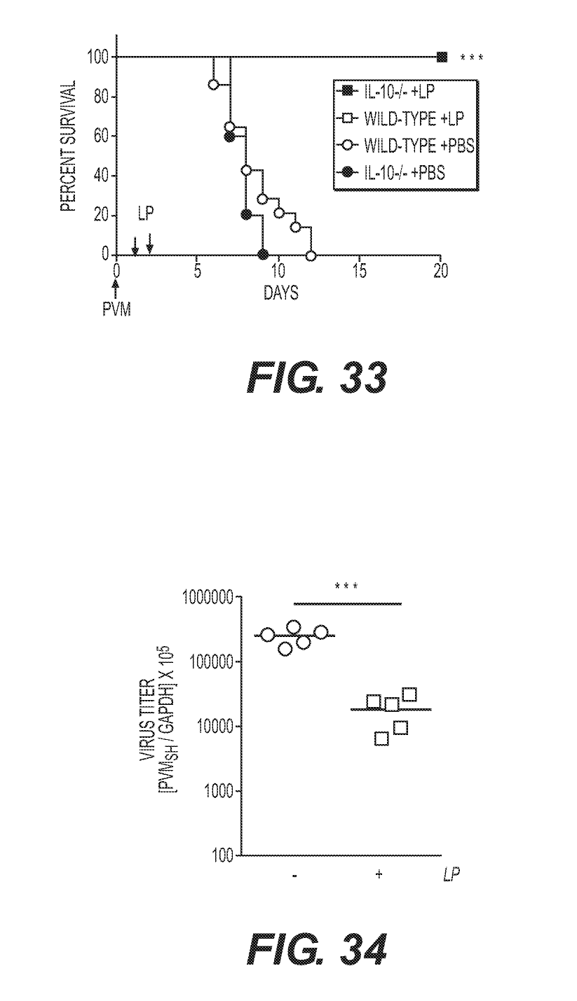

Eight week old, single gene deleted interleukin-10 (IL-10^{-/-}) mice were inoculated with L. plantarum, LP-F0 (50 uL of 2×10¹⁰ cells/mL) or PBS on days +1 and +2 after PVM challenge. IL-10^{-/-} mice primed with L. plantarum were fully protected from the lethal sequelae of PVM infection unlike to their counterparts that were primed with diluent (pbs/bsa) only, FIG. 33 (***p<0.001 log-rank).

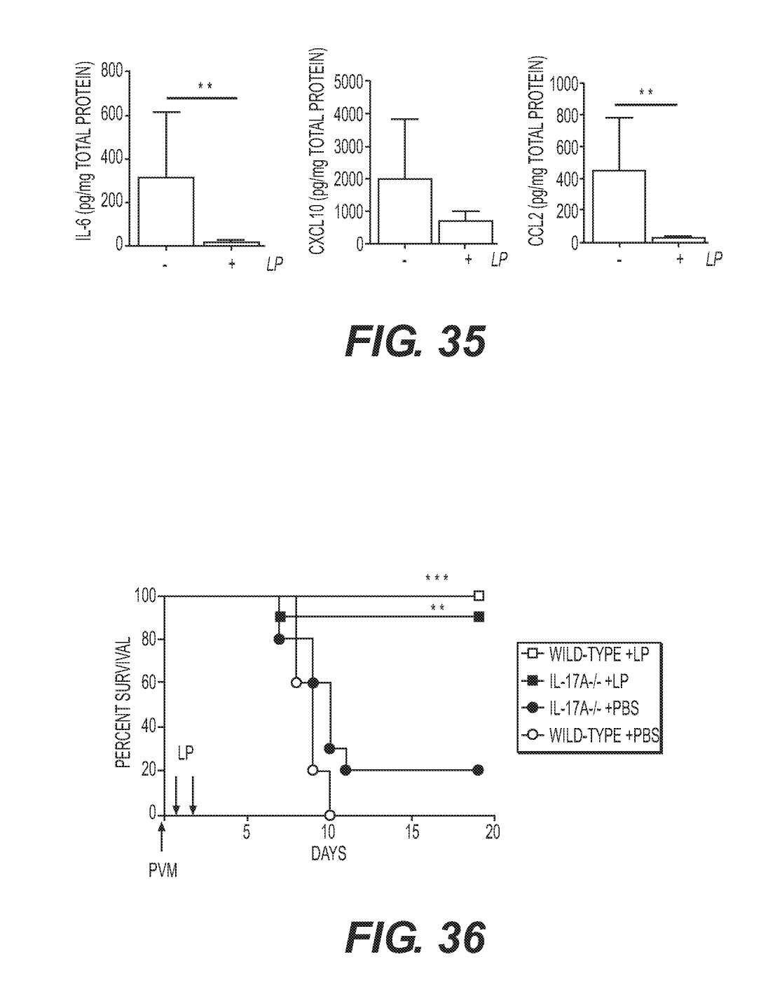

Analogous to their wild type (BALB/c) counterparts (FIGS. 3 and 4), the administration of L. plantarum to IL-10^{-/-} mice on days +1 and +2 after PVM challenge results in diminished virus recovery from lung tissue, FIG. 34 (***p<0.001, Mann-Whitney U-test) and prominent suppression of cytokines CCL2, CXCL10, and IL-6, FIG. 35 (**p<0.01, Mann-Whitney U-test). Eight week old, IL17A^{-/-} mice were inoculated with L. plantarum, LP-F0 (50 uL of 2×10¹⁰ cells/mL) or PBS on days +1 and +2 after PVM challenge. Analogous to their wild (C57BL/6) counterparts, IL17A^{-/-} primed mice were fully protected from the lethal sequelae of PVM infection unlike to their counterparts that were primed with diluent (pbs/bsa) only, FIG. 36 (**p<0.01; ***p<0.001 log-rank).

### Example 22

Priming with L. plantarum Results in Profound Suppression of Specific Virus-Induced Proinflammatory Mediators.

BALB/c mice were inoculated intranasally on day −14 and again on day −7 with 10⁹ cfu of live L. plantarum in pbs/bsa (50 μL of 2×10¹⁰ cells/mL) or pbs/bsa diluent control alone. In this experiment, mice are then challenged 21 days later (on day +14) with an otherwise lethal dose of PVM strain J3666 or vehicle only. RNA was isolated from whole lung tissue (pooled from 6 mice per group) and was subjected to whole genome microarray analysis; differential expression of thirty-one (31) soluble proinflammatory mediators identified in this experiment is featured in Table 1. As shown, PVM infection in BALB/c mice results in the increased expression of transcripts encoding numerous CC and CXC chemokines and acute phase reactants such as serum amyloid A1 and A3 and other soluble proinflammatory mediators.

TABLE 1

| Gene | Entrez | | PBS/PVM^{a} vs. | LP/PVM^{b} vs. | LP/PVM^{c} vs. |
|---|---|---|---|---|---|
| Symbol | Gene | Description | VEH | VEH | PVM |
| Gzmb | 14939 | granzyme B | 309.0 | 83.2 | |
| Il6 | 16193 | interleukin 6 | 231.5 | 2.2* | −105.0 |
| Ccl2 | 20296 | chemokine (C-C motif) ligand 2 | 225.3 | 16.6* | −13.6 |
| Cxcl10 | 15945 | chemokine (C-X-C motif) ligand 10 | 75.2 | 6.8 | −11.0 |
| Lcn2 | 16819 | lipocalin 2 | 68.0 | 20.5 | −3.3 |
| Cxc19 | 17329 | chemokine (C-X-C motif) ligand 9 | 60.8 | 8.1* | |
| Cxc12 | 20310 | chemokine (C-X-C motif) ligand 2 | 56.7 | 2.8* | −20.0 |
| Saa3 | 20210 | serum amyloid A 3 | 50.3 | 25.1 | |
| Saa1 | 20208 | serum amyloid A 1 | 50.1 | −1.7* | −86.7 |
| Cc17 | 20306 | chemokine (C-C motif) ligand 7 | 47.2 | 34* | −13.8 |
| Cxcl1 | 14825 | chemokine (C-X-C motif) ligand 1 | 47.1 | 14.7 | |
| Cxcl11 | 56066 | chemokine (C-X-C motif) ligand 11 | 32.8 | 1.6* | −21.1 |
| Cel12 | 20293 | chemokine (C-C motif) ligand 12 | 13.1 | 8.6* | |
| Il1rn | 16181 | interleukin 1 receptor antagonist | 9.8 | 4.3* | |
| Lgals3bp | 19039 | lectin, galactoside-binding, soluble, | 9.3 | 8.3 | |
| | | 3 binding pro | | | |
| Thbs1 | 21825 | thrombospondin 1 | 8.9 | 1.6* | −5.5 |
| S100a8 | 20201 | S100 calcium binding protein A8 | 7.3 | −1.0* | |
| | | (calgranul in A) | | | |
| Csf1 | 12977 | colony stimulating factor 1 (macrophage) | 7.2 | 3.6* | |
| S100a9 | 20202 | S100 calcium binding protein A9 | 6.6 | −1.7* | −11.1 |
| | | (calgranul in B) | | | |
| Lgals9 | 16859 | lectin, galactose binding, soluble 9 | 6.2 | 3.0* | |
| Cxcl13 | 55985 | chemokine (C-X-C motif) ligand 13 | 4.0 | 3.9* | |
| Ccl5 | 20304 | chemokine (C-C motif) ligand 5 | 3.1 | 2.9* | |
| Ccl9 | 20308 | chemokine (C-C motif) ligand 9 | 3.0 | 4.6 | |
| Cxcl16 | 66102 | chemokine (C-X-C motif) ligand 16 | 3.0 | 1.7* | |
| Lgals3 | 16854 | lectin, galactose binding, soluble 3 | 2.6 | 2.4 | |
| Hmgbl | 15289 | high mobility group protein B1-like | −2.2 | −1.1* | 2.0 |
| Cxcl15 | 20309 | chemokine (C-X-C motif) ligand 15/IL8 | −2.2 | 1.3* | 2.8 |
| Il33 | 77125 | interleukin 33 | −2.9 | 1.9* | 5.4 |
| Cxcl12 | 20315 | chemokine (C-X-C motif) ligand 12 | −5.9 | −2.1* | 2.8 |
| Cc18 | 20307 | chemokine (C-C motif) ligand 8 | 3.4* | 16.7 | 5.0 |
| Cc16 | 20305 | chemokine (C-C motif) ligand 6 | −1.3* | 3.8 | 4.9 |

^{a}mice primed with diluent (PBS) and inoculated with PVM vs. mice primed with diluent (pbs/bsa) and inoculated with vehicle (VEH).
^{b}mice primed with L. plantarum (LP) and inoculated with PVM vs. mice primed with diluent (PBS) and inoculated with vehicle (VEH); *values not significant (>0.05) over PBS/VEH
^{c}mice primed with L. plantarum (LP) and inoculated with PVM vs. mice primed with diluent (PBS) and inoculated with PVM; only statistically significant differences are shown.

Priming of the respiratory tract with L. plantarum prior to virus infection results in a broad-spectrum anti-inflammatory profile. Among those inflammatory mediators with expression most profoundly suppressed was virus-induced interleukin (IL)-6, with expression diminished 105-fold in response to L. plantarum priming. Other chemokines that respond with profound suppression include CCL2, CXCL10, CXCL2 and CXCL11, which undergo 11, 14, 20 and 21-fold reduced expression, respectively. Although the predominant effect of priming prior to viral infection is anti-inflammatory, several of the 31 pro-inflammatory chemokines experience no significant differential expression. Thus, there appears to be some specificity in the anti-inflammatory program modulated by L. plantarum at the respiratory epithelium.

1-100. (canceled)

101. A pharmaceutical composition comprising composite particles comprising one or more species of Lactobacillus bacteria and an excipient, wherein each composite particle comprises a percentage loading of Lactobacillus ranging from about 1% w/w to about 97% w/w.

102. A pharmaceutical composition according to claim 101 wherein said Lactobacillus is heat inactivated.

103. A pharmaceutical composition according to claim 102, wherein said heat inactivated Lactobacillus is greater than 95% whole cell.

104. A pharmaceutical composition according to claim 101, wherein the excipient is selected from one or more of trehalose, lactose, leucine, di-leucine, tri-leucine, dextran, cyclodextran, maltose, sucrose, glucose, sorbitol, erythritol, mannitol, dextrose, maltitol, maltose, mannilol, raffinose, galactose, xylose, ribose, xylitol, tryptophan, tyrosine, phenylalanine, and maltodextrin.

105. A pharmaceutical composition according to claim 104, wherein the excipient comprises tri-leucine, and the composite particles comprise a ratio of Lactobacillus to tri-leucine of greater than about 0.01% w/w to about 10% w/w.

106. A pharmaceutical composition according to claim 101 comprising: from about 40 to about 60% Lactobacillus; from about 40 to about 60% w/w of excipient wherein the excipient is trehalose; wherein the Lactobacillus is heat inactivated; and wherein the Lactobacillus is whole cell.

107. A pharmaceutical composition according to claim 103 wherein the Lactobacillus species is selected from the group consisting of L. acetotolerans, L. acidifarinae, L. acidipiscis, L. acidophilus, L. agilis, L. algidus, L. alimentarius, L. amylolyticus, L. amylophilus, L. amylotrophicus, L. amylovorus, L. animalis, L. antri, L. apodemi, L. aviaries, L. bifidus, L. bifermentans, L. brevis, L. buchneri, L. bulgaricus, L. camelliae, L. casei, L. catenaformis, L. ceti, L. coleohominis, L. collinoides, L. composti, L. concavus, L. coryniformis, L. crispatus, L. crustorum, L. curvatus, L. delbrueckii subsp. Delbrueckii, L. delbrueckii subsp. Bulgaricus, L. delbrueckii subsp. Lactis, L. dextrinicus, L. diolivorans, L. equi, L. equigenerosi, L. farraginis, L. farciminis, L. fermentii, L. fermentum, L. fornicalis, L. fructivorans, L. frumenti, L. fuchuensis, L. gallinarum, L. gasseri, L. gastricus, L. ghanensis, L. graminis, L. hammesii, L. hamster, L. harbinensis, L. hayakitensis, L. helveticus, L. hilgardii, L. homohiochii, L. iners, L. ingluviei, L. intestinalis, L. jensenii, L. johnsonii, L. kalixensis, L. kefiranofaciens, L. kefiri, L. kimchii, L. kitasatonis, L kunkeei, L. lactis, L. leichmannii, L. lindneri, L. malefermentans, L. mail, L. manihotivorans, L. mindensis, L. mucosae, L. murinus, L. nagelii, L. namurensis, L. nantensis, L. oligofermentans, L. oris, L. panis, L. pantheris, L. parabrevis, L. parabuchneri, L. paracasei, L. paracollinoides, L. parafarraginis, L. parakefiri, L. paralimentarius, L. paraplantarum, L. pentosus, L. perolens, L. plantarum, L. pontis, L. psittaci, L. rennin, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. thailandensis, L. thermophilus, L. ultunensis, L. vaccinostercus, L. vaginalis, L. versmoldensis, L. vini, L. vitulinus, L. zeae, and L. zymae.

108. A pharmaceutical composition according to claim 107 wherein the Lactobacillus is a single species comprising L. plantarum.

109. A pharmaceutical composition according to claim 108 wherein the L. plantarum comprises a single strain selected from the group consisting of ATCC 10241, ATCC 14431, ATCC 39268, ATCC 21028, ATCC 55324, ATCC 39542, ATCC 14917, ATCC 700211, ATCC BAA-793, ATCC 4008, ATCC 8014, ATCC 10012, ATCC 49445, ATCC 53187, ATCC 700210, ATCC BAA-171, DSMZ 10492, DSMZ 1055, DSMZ 12028, DSMZ 24624, DSMZ 2648, DSMZ 6872 and DSMZ 16365.

110. A device for delivering a pharmaceutical composition comprising dry powder composite particles comprising Lactobacillus bacteria and an excipient, wherein the composite particles have a mass median aerodynamic diameter (MMAD) ranging from about 20μηη to about 30μηη.

111. A device according to claim 110 which is an inhaler.

112. A device according to claim 110 which is an intranasal dry powder delivery device.

113. A method of preventing or treating a viral infection or the symptoms thereof in a subject comprising administering to the subject a composition comprising one or more species of Lactobacillus bacteria.

114. A method according to claim 113 comprising administering to the subject a composition comprising a single species of Lactobacillus bacteria.

115. A method according to claim 114 comprising administering to the subject a composition comprising L. plantarum.

116. A method according to claim 115 comprising administering to the subject a composition comprising a single strain of L. plantarum which is selected from the group consisting of ATCC 10241, ATCC 14431, ATCC 39268, ATCC 21028, ATCC 55324, ATCC 39542, ATCC 14917, ATCC 700211, ATCC BAA-793, ATCC 4008, ATCC 8014, ATCC 10012, ATCC 49445, ATCC 53187, ATCC 700210, ATCC BAA-171, DSMZ 10492, DSMZ 1055, DSMZ 12028, DSMZ 24624, DSMZ 2648, DSMZ 6872 and DSMZ 16365.

117. A method according to claim 115 comprising administering to the subject a composition comprising a single strain of plant-derived L. plantarum which is selected from the group consisting of ATCC 10241, ATCC 14431, ATCC 55324, ATCC 39542, ATCC 14917, ATCC 700211, ATCC 53187, ATCC BAA-171, DSMZ 10492, DSMZ 24624, DSMZ 2648, and DSMZ 16365.

118. A method according to claim 113 comprising administering to the subject a single dose of Lactobacillus bacteria.

119. A method according to claim 113 comprising administering to the subject at least 2 doses of Lactobacillus bacteria.

120. A method of preventing or treating a viral infection or the symptoms thereof in a subject comprising administering to the subject a loading dose of 2 or more doses of Lactobacillus bacteria, followed by subsequent weekly, bi-weekly or monthly doses.

121. A method according to claim 120 comprising administering to the subject the loading dose in a week, followed by subsequent weekly doses.

122. A method according to claim 120 comprising administering to the subject the loading dose over 2 weeks, followed by subsequent bi-weekly doses.

123. A method according to claim 120 comprising administering to the subject the loading dose over 2 weeks followed by subsequent monthly doses.

124. A method of preventing or treating a viral infection or the symptoms thereof in a subject comprising administering to the subject one or more doses of Lactobacillus bacteria, wherein at least one dose of Lactobacillus is administered between 1 and 7 days prior to, or between 1 and 7 days after, viral exposure.

125. A method according to claim 124, wherein at least 2 doses of Lactobacillus are administered between 1 and 7 days prior to, or between 1 and 7 days after, viral exposure.

126. A method according to claim 125, wherein at least 2 doses of Lactobacillus are administered between 1 and 2 days after viral exposure.

127. A method according to claim 125, wherein at least 2 doses of Lactobacillus are administered at an interval of 7 days prior to viral exposure.

128. A method according to claim 125, wherein at least 2 doses of Lactobacillus are administered at an interval of 2 days prior to viral exposure.

129. A method according to claim 113 comprising administering to the subject a composition comprising one or more species of Lactobacillus bacteria to suppress virus-induced inflammation and/or virus-induced cytokine induction.

130. A method of preventing or treating a secondary respiratory bacterial infection following an initial respiratory viral infection in a subject comprising administering to the subject a composition comprising one or more species of Lactobacillus bacteria.

131. A method of preventing or treating at least one symptom of a cold or flu in a subject in need thereof comprising administering to the subject a composition comprising one or more species of Lactobacillus bacteria.
